(19) United States
(12) Patent Application Publication
(10) Pub. No.: US 2024/0206937 A1
(43) Pub. Date: Jun. 27, 2024

(54) TUMOR ABLATION TOOLS AND TECHNIQUES

(71) Applicant: SNIPE MEDICAL LTD, Tel Aviv (IL)

(72) Inventors: Yuval TAFF, Tel Aviv (IL); Ben OMRI, Ashkelon (IL)

(21) Appl. No.: 18/288,335

(22) PCT Filed: May 10, 2022
(86) PCT No.: PCT/IB2022/054313

§ 371 Date: Oct. 25, 2023

Related U.S. Application Data
(60) Provisional application No. 63/191,992, filed on May 22, 2021.

(51) Int. Cl. A61B 18/00

(52) U.S. Cl. CPC A61B 18/00; A61B 2018/00083; A61B 2018/0016; A61B 2018/00184; A61B 2018/00541; A61B 2018/00577; A61B 2018/00642; A61B 2018/0072; A61B 2018/00732; A61B 2018/00767; A61B 2018/00839

(57) ABSTRACT

An apparatus for use with a tumor comprises a tumor-ablating device that has a distal region that comprises (i) an inner shaft, comprising: (a) a first bioimpedance-sensing electrode, and (b) a first electroporation electrode, mounted at a fixed position proximally from the first bioimpedance-sensing electrode, and (ii) an outer shaft within which the inner shaft is coaxially disposed, the outer shaft comprising: (a) a second bioimpedance-sensing electrode, and (b) a second electroporation electrode, mounted at a fixed position distally from the second bioimpedance-sensing electrode. The inner shaft and the outer shaft are telescopically slidable with respect to each other in a manner that changes a first axial distance between the first electroporation electrode and the second electroporation electrode. Other embodiments are also described.

### CROSS-REFERENCES TO RELATED APPLICATIONS

The present application claims priority to U.S. Provisional Patent Application 63/191,992 to Taff, filed May 22, 2021, and entitled “Adjustable electrode configurations for tumor ablation,” which is incorporated herein by reference.

### FIELD OF THE INVENTION

The present application relates to the field of medical devices, particularly devices for tumor ablation.

### BACKGROUND

Electric field pulses of high intensity and short duration can cause tissue ablation in a process called irreversible electroporation (IRE). IRE has been described in the art for treatment of tumors in various locations in the body and most commonly inserted percutaneously.

J. Ricke et al. (The ALICE Trial, Cardiovasc Intervent Radiol (2015) 38:401-408) have described the use of IRE for percutaneous treatment of lung tumors. The authors failed to demonstrate the efficacy of this technique, with the conclusion that the variance in electric properties between the tumor and surrounding tissue together with inaccurate positioning of the electrodes caused improper electric field distribution inside the target tissue.

### SUMMARY OF THE INVENTION

The present disclosure relates to methods and systems for determining the boundaries of a tumor using electrical impedance, in order to position a treatment applicator within the boundaries of the tumor. For example, the treatment applicator may comprise treatment electrodes, such as electroporation electrodes. It may be advantageous to position the treatment electrodes within the boundaries of the tumor, e.g. to focus the treatment on tumor tissue rather than surrounding tissue. It may further be advantageous to position the treatment electrodes close to the boundaries of the tumor in order to treat the entire tumor—e.g. in order to avoid leaving tumor cells near the boundaries of the tumor untreated.

For some applications, due to differences in tissue properties between the tumor itself and its surrounding tissue, it may be possible to determine the boundaries of the tumor by sensing bioimpedance. For example, healthy lung tissue typically has high bioimpedance (e.g. due to the air-filled structures of the parenchyma and its alveoli), whereas a lung tumor typically has substantially lower bioimpedance. Thus, bioimpedance-sensing electrodes positioned close to, and/or within, the tumor may be able to provide information regarding the boundaries of a tumor—particularly a lung tumor. Furthermore, this difference in bioimpedance between the tumor and its surrounding tissue may allow for successful electroporation of the tumor, while minimizing collateral damage to the surrounding tissue. For example, the relatively high conductivity of the tumor, relative to the surrounding tissue, may facilitate conduction of an electroporation pulse throughout the tumor but not into the surrounding tissue.

For some applications, a device having electroporation electrodes at a distal region of the device is advanced into a patient. For applications in which the tumor is a lung tumor, the device may be advanced into the patient using transbronchially e.g. via the nose or mouth of the patient, down the trachea, and into an airway of the patient. For some applications, the advancement is performed percutaneously (e.g. transluminally or transthoracically).

For some applications, the device includes one or more bioimpedance-sensing electrodes at its distal region. Once the distal region has been positioned at the tumor, the boundaries of the tumor can be determined using the bioimpedance-sensing electrodes. For some applications, due to the differences in tissue properties between the tumor and its surrounding tissue e.g. as described hereinabove, the device (e.g. an extracorporcal control unit thereof), can determine (or can facilitate determination of) the boundaries of the tumor by moving (or facilitating movement of) the bioimpedance-sensing electrodes with respect to the tumor, and sensing the bioimpedance in the different positions. For example, the bioimpedance-sensing electrodes may be advanced into and/or out of the tumor so that the precise position of the transition between inside and outside (i.e. the boundary) can be identified. Based on the identified boundary, a treatment applicator (e.g. an electroporation electrode) may be accurately positioned within the tumor (e.g. close to the boundary), and used to apply treatment, such as an electroporation pulse, to the tumor. For some applications, the treatment is applied while the bioimpedance-sensing electrodes remain in place—e.g. at, just inside of, or just outside of the boundary. For some applications, the treatment is applied after moving the bioimpedance-sensing electrodes. For some applications, the bioimpedance-sensing electrodes also serve as the treatment applicator—e.g. as electroporation electrodes between which an electroporation pulse is applied.

For some applications, the treatment applicator is in a fixed and/or has a known position with respect to the bioimpedance-sensing electrode, such that once the boundaries of the tumor have been determined, the treatment applicator is positioned just within the boundaries of the tumor. For some applications, determining the boundaries of the tumor using the bioimpedance-sensing electrode automatically positions the treatment applicator within the tumor. For example, a device may be structured such that positioning a bioimpedance-sensing electrode at, or just outside of, a boundary of the tumor may automatically position a corresponding electroporation electrode just inside of the boundary. For some applications, once the boundaries of the tumor have been determined, an operator can move the treatment applicator into and/or within the tumor, using the known relative positions between the treatment applicator and the bioimpedance-sensing electrode.

For some applications, the bioimpedance-sensing electrode can be used to detect a boundary of the tumor and provide an alert indicating, for example, that no further advancement is required (or desired).

For some applications, the treatment applicator (e.g. electroporation electrodes) is positioned axially between the bioimpedance-sensing electrodes, such that identifying that the bioimpedance sensing electrodes are at (e.g. just outside of) the boundaries of tumor may automatically verify that the electroporation electrodes are just within the boundaries.

For some applications, the bioimpedance-sensing electrodes may be used to determine whether a lesion or growth is a cancerous tumor—e.g. to facilitate diagnosis. For example, the bioimpedance of a cancerous tumor may be different to that of a non-cancerous lesion or growth.

For some applications, it may be possible to determine whether the bioimpedance-sensing electrode(s) (and therefore the electroporation electrode(s)) are positioned within the tumor by monitoring the bioimpedance measured at the bioimpedance-sensing electrode(s) over the breathing cycle of the patient. For example, the bioimpedance values of the parenchyma of the lung may vary (e.g. oscillate) during the breathing cycle (e.g. impedance decreasing during exhalation and increasing during inhalation) whereas a tumor may have a more consistent bioimpedance value throughout the breathing cycle. For some applications, receiving a signal with a magnitude of oscillation above a threshold magnitude is indicative that the tissue in which the bioimpedance-sensing electrode is disposed is lung tissue (e.g. parenchyma of the lung) rather than tumor tissue.

For some applications, the frequency of the oscillations sensed at the tumor by the bioimpedance-sensing electrode is compared with the frequency of the breathing cycle of the patient to ensure that the sensed oscillations are reflective of the breathing cycle, and do not arise from other factors (e.g. the pulse of the patient). This may therefore provide further validation to the operator regarding whether a particular tissue is a tumor, and/or the position of the bioimpedance-sensing electrode(s) with respect to the tumor.

There is therefore provided, in accordance with some applications, a method comprising (i) receiving information indicative of bioimpedance of a tissue in a lung of a subject (optionally including information indicative of oscillation of the bioimpedance, such as magnitude and/or frequency of the oscillation), and (ii) responsively, determining (e.g. diagnosing) whether the tissue is a tumor and/or is cancerous.

In general, some embodiments of the present disclosure include an apparatus configured from at least two electrodes that are moveable in reference to each other.

In some embodiments the electrodes can be electrically and/or mechanically connected to serve as a single monopolar electrode, with an external grounding pad. In some embodiments, the single monopolar electrode may be equipped with one or more bioimpedance sensing electrodes.

In some embodiments, one or more electrodes (i.e. inner electrodes) are configured to pass through a second electrode (outer electrode). In some embodiments, the electrodes are connected to an energy source configured to apply high voltages required to induce electroporation (reversible and irreversible).

In some embodiments, the apparatus is configured to be delivered via bronchoscopy procedure to treat tumors that are situated in the lungs or airways.

In some embodiments, the electrodes are curved or deflectable.

In some embodiments, the device may also include sensors mounted near or on one or more of the electrodes to enable their accurate positioning inside the tumor.

In some embodiments, the sensors are in the form of electrodes and measure the impedance values between the sensors and treatment electrodes

In some embodiments, subsequently to introducing the outer electrode into the tumor the inner electrode is inserted through the outer electrode into the tumor as well. Voltage is then applied between the inner electrode and outer electrode. The voltage causes electroporation (either reversible or irreversible) in the vicinity of the electrode. Electrodes may then be adjusted to cover additional segments of the tumor. In some embodiments the inner electrode may be inserted into the tumor prior to inserting the outer electrode and the outer electrode may be inserted over the inner electrode into the tumor.

In some embodiments, a sensor mounted proximal to the outer electrode is used to verify that the outer electrode has entered the tumor.

In some embodiments, a sensor mounted distal to the inner electrode is used to verify that the inner electrode has reached the distal edge of the tumor.

In some embodiments, covering additional segments of the tumor is accomplished by moving the inner electrode.

In some embodiments, covering additional segments of the tumor is accomplished by using a curved or deflectable electrode.

There is therefore provided, in accordance with some applications, an apparatus for ablating a tumor, the apparatus including a device having a distal region.

For some applications, the device includes a bioimpedance-sensing electrode at the distal region and a treatment applicator, adapted to ablate tissue of the tumor, and positionally fixed at the distal region with respect to the bioimpedance-sensing electrode.

For some applications, the device includes a shaft to which the bioimpedance-sensing electrode is attached, the shaft being advanceable:
to a first position in which the bioimpedance-sensing electrode is disposed within the tumor, and
to a second position in which the bioimpedance-sensing electrode is disposed outside of the tumor, and the treatment applicator is disposed within the tumor.

For some applications, in the first position of the shaft, the treatment applicator is disposed outside of the tumor.

For some applications, the distal region defines a tissue-piercing tip.

For some applications, the distal region is curvable within the tumor.

For some applications, the device is a first device, and the apparatus further includes at least one more additional device, each additional device having a distal region including a treatment applicator and a bioimpedance-sensing electrode.

For some applications, the treatment applicator is attached to the shaft, proximally from the bioimpedance-sensing electrode.

For some applications, the treatment applicator is attached to the shaft, distally to the bioimpedance-sensing electrode.

For some applications, the shaft is flexible.

For some applications, the shaft is rigid.

For some applications:
the treatment applicator includes an electroporation electrode,
the shaft is a first shaft, the electroporation electrode being disposed on the first shaft,
the device further includes a second shaft, and
the first shaft and the second shaft are reversibly movable with respect to each other by the first shaft being axially slidable with respect to the second shaft.

For some applications:
the first shaft is an inner shaft,
the second shaft is an outer shaft, slidable over the inner shaft, and
the device is configured such that an effective length of the electroporation electrode is adjustable by sliding the outer shaft over the electroporation electrode.

For some applications, the bioimpedance-sensing electrode is disposed on the first shaft, distally from the electroporation electrode.

For some applications, the device further includes an other bioimpedance-sensing electrode, disposed on the second shaft.

For some applications:
the tumor is a tumor disposed in a lung of a subject, and
the apparatus is configured to determine a boundary of the lung tumor via sensing performed by the bioimpedance-sensing electrode.

For some applications, the apparatus includes a bronchoscope, transbronchially advanceable to the lung, the shaft deliverable to the tumor via the bronchoscope.

For some applications, the apparatus further includes a remote electrode, and the apparatus is configured to sense bioimpedance between the remote electrode and the bioimpedance-sensing electrode.

For some applications, the remote electrode is a skin electrode.

For some applications, the treatment applicator:
includes an electroporation electrode, and
is adapted to apply an electroporation pulse to the tumor using the electrode.

For some applications, the apparatus further includes a remote electrode, and the apparatus is configured to apply the electroporation pulse between the remote electrode and the electroporation electrode.

For some applications, the apparatus is configured to sense bioimpedance at the tumor by sensing bioimpedance between the electroporation electrode and the bioimpedance-sensing electrode.

For some applications:
the electroporation electrode is a first electrode,
the treatment applicator further includes a second electroporation electrode, and
the apparatus is configured to apply the electroporation pulse between the first electrode and the second electrode.

For some applications, the first and second electroporation electrodes are mounted on the distal region, the distal region being reversibly lengthenable in a manner that changes an axial distance between the first electrode and the second electrode.

For some applications, the distal region includes a telescopic assembly having:
a distal portion having the first electroporation electrode disposed thereon, and
a proximal portion having the second electroporation electrode disposed thereon, the distal region being reversibly lengthenable by the distal portion and the proximal portion being axially slid with respect to each other.

For some applications, the bioimpedance-sensing electrode is disposed on the distal portion, distally to the first electroporation electrode.

For some applications, the device further includes a second bioimpedance-sensing electrode, disposed on the proximal portion, proximally from the second electroporation electrode.

For some applications:
the shaft is a first shaft, the first electroporation electrode being disposed on the first shaft,
the device further includes a second shaft, the second electroporation electrode being disposed on the second shaft, and
the first shaft and the second shaft are reversibly movable with respect to each other by the first shaft being slidable through the second shaft.

For some applications, at the distal region, the first shaft and the second shaft fork with respect to each other.

For some applications, each of the first shaft and the second shaft are adapted to access the tumor from independent tumor-access sites.

For some applications, the second shaft defines a side-port out of which the first shaft is advanceable.

For some applications, the first shaft is curvable with respect to the second shaft.

For some applications, the first shaft is advanceable out of a distal end of the second shaft.

For some applications, the apparatus includes a control unit, adapted to receive a signal from the bioimpedance-sensing electrode, and to responsively provide an output indicative of bioimpedance of tissue adjacent the bioimpedance-sensing electrode.

For some applications, the control unit is adapted to identify a change in the signal over at least a portion of a breathing cycle of the patient, the output being indicative of a change in bioimpedance of the tissue adjacent the bioimpedance-sensing electrode over at least the portion of the breathing cycle.

For some applications, the control unit is configured to, responsively to the signal, output information indicative of the position of the bioimpedance-sensing electrode with respect to the tumor.

For some applications:
the device includes a terminal, electrically connected to the bioimpedance-sensing electrode via a conductor extending along the shaft, and
the control unit is electrically connectable to the bioimpedance-sensing electrode by electrically connecting the control unit to the terminal.

For some applications:
the treatment applicator is configured to apply an electroporation pulse to the tumor, and
the control unit includes a power generator, electrically connectable to the treatment applicator, and adapted to drive the treatment applicator to apply the electroporation pulse.

For some applications, the treatment applicator is positionally fixed with respect to the bioimpedance-sensing electrode at a distance of 10 mm or less.

For some applications, the treatment applicator is positionally fixed with respect to the bioimpedance-sensing electrode at a distance of 5 mm or less.

There is further provided, in accordance with some applications, an apparatus for ablating a tumor in a lung of a subject.

For some applications, the apparatus includes a device that includes:
a shaft;
a bioimpedance-sensing electrode attached to a distal region of the shaft; and
an electroporation electrode, attached to the distal region of the shaft at a fixed distance from the bioimpedance-sensing electrode.

For some applications, the distal region of the shaft is advanceable into a position within the lung in which:
the electroporation electrode is disposed within a boundary of the tumor, and
the bioimpedance-sensing electrode is disposed outside of the boundary of the tumor.

For some applications, the apparatus is configured to determine the boundary of the lung tumor via sensing performed by the bioimpedance-sensing electrode.

For some applications, the apparatus includes a bronchoscope, transbronchially advanceable to the lung, the shaft deliverable to the tumor via the bronchoscope.

For some applications, the distal region defines a tissue-piercing tip.

For some applications, the distal region is curvable within the tumor.

For some applications, the electroporation electrode is attached to the shaft proximally from the bioimpedance-sensing electrode.

For some applications, the electroporation electrode is attached to the shaft distally to the bioimpedance-sensing electrode.

For some applications, the shaft is flexible.

For some applications, the shaft is rigid.

For some applications, the apparatus further includes a remote electrode, and the apparatus is configured to sense bioimpedance between the remote electrode and the bioimpedance-sensing electrode.

For some applications, the remote electrode is a skin electrode.

For some applications, the electroporation electrode is adapted to apply an electroporation pulse to the tumor.

For some applications, the apparatus further includes a remote electrode, and the apparatus is configured to apply the electroporation pulse between the electroporation electrode and the remote electrode.

For some applications, the apparatus is configured to sense bioimpedance at the tumor by sensing bioimpedance between the electroporation electrode and the bioimpedance-sensing electrode.

For some applications:
the electroporation electrode is a first electroporation electrode,
the device further includes a second electroporation electrode, and
the apparatus is configured to apply the electroporation pulse between the first electroporation electrode and the second electroporation electrode.

For some applications:
the shaft is a first shaft, the first electroporation electrode being disposed on the first shaft,
the device further includes a second shaft, the second electroporation electrode being disposed on the second shaft, and
the first shaft and the second shaft are reversibly movable with respect to each other by the first shaft being slidable through the second shaft.

For some applications, at a distal region of the device, the first shaft and the second shaft fork with respect to each other.

For some applications, each of the first shaft and the second shaft are adapted to access the tumor from independent tumor-access sites.

For some applications, the bioimpedance-sensing electrode is disposed on the first shaft, distally to the first electroporation electrode.

For some applications, the device further includes a second bioimpedance-sensing electrode, disposed on the second shaft, proximally from the second electroporation electrode.

For some applications, the second shaft defines a side-port out of which the first shaft is advanceable.

For some applications, the first shaft is curvable with respect to the second shaft.

For some applications, the first shaft is advanceable out of a distal end of the second shaft.

For some applications, the apparatus includes a control unit, adapted to receive a signal from the bioimpedance-sensing electrode, and to responsively provide an output indicative of bioimpedance of tissue adjacent the bioimpedance-sensing electrode.

For some applications, the control unit is adapted to identify a change in the signal over at least a portion of a breathing cycle of the patient, the output being indicative of a change in bioimpedance of the tissue adjacent the bioimpedance-sensing electrode over at least the portion of the breathing cycle.

For some applications, the control unit is configured to, responsively to the signal, provide an output indicative of the position of the bioimpedance-sensing electrode with respect to the tumor.

For some applications:
the device includes a terminal, electrically connected to the bioimpedance-sensing electrode via a conductor extending along the shaft, and
the control unit is electrically connectable to the bioimpedance-sensing electrode by electrically connecting the control unit to the terminal.

For some applications, the control unit is adapted to provide an electrical current to generate the signal.

For some applications:
the electroporation electrode is configured to apply an electroporation pulse to the tumor, and
the control unit includes a power generator, electrically connectable to the electroporation electrode, and adapted to drive the electroporation electrode to apply the electroporation pulse.

For some applications, the fixed distance is 10 mm or less.

For some applications, the fixed distance is 5 mm or less.

For some applications, the shaft is a first shaft, and the apparatus further includes at least a second shaft, the second shaft having a distal region including an electroporation electrode and a bioimpedance-sensing electrode.

For some applications:
the device is a first device, the first shaft being a shaft of the first device, and
the apparatus further includes a second device, the second shaft being a shaft of the second device.

There is further provided, in accordance with some applications, a method for ablating a tumor in a tissue of a subject, the tumor having a boundary, the method including:
advancing a distal region of a tumor-ablating device into the tissue, the distal region including an electrode mounted thereon;
using the distal region of the device, sensing bioimpedance of the tissue.

For some applications, the electrode is positioned within the boundary of the tumor responsively to the sensed bioimpedance.

For some applications, an electroporation pulse is applied to the tumor using the electrode while the electrode remains within the boundary.

For some applications:
sensing bioimpedance of the tissue includes sensing changes in the bioimpedance of the tissue over at least a portion of a breathing cycle of the subject, and
responsively to the sensed changes in the bioimpedance, positioning the electrode within the boundary of the tumor.

For some applications:
the electrode is disposed on a first shaft of the device,
the device further includes a second shaft, and
the method further includes, subsequently to positioning the electrode within the boundary of the tumor, axially sliding the first shaft with respect to the second shaft.

For some applications:
the first shaft is an inner shaft,
the second shaft is an outer shaft, slidable over the inner shaft, and axially sliding the first shaft with respect to the second shaft includes axially sliding the outer shaft over the inner shaft in a manner that adjusts an effective length of the electrode.

For some applications:
the electrode is an electroporation electrode,
the distal region further includes a bioimpedance-sensing electrode,
sensing the bioimpedance using the distal region of the device includes sensing the bioimpedance using the bioimpedance-sensing electrode, and
positioning the electroporation electrode within the boundary of the tumor includes moving the distal region of the device through the tumor until the bioimpedance-sensing electrode exits the tumor.

For some applications:
sensing the bioimpedance using the bioimpedance-sensing electrode includes sensing the bioimpedance using the bioimpedance-sensing electrode while the bioimpedance-sensing electrode is electrically connected to a control unit, the control unit being configured to provide an alert in response to a change in bioimpedance detected via the bioimpedance-sensing electrode upon the exit of the bioimpedance-sensing electrode from the tumor, and
positioning the electrode within the boundary of the tumor includes positioning the electrode within the boundary, responsively to the alert.

For some applications, the bioimpedance-sensing electrode is disposed distally to the electroporation electrode, and moving the distal region of the device through the tumor until the bioimpedance-sensing electrode exits the tumor includes moving the distal region of the device distally through the tumor until the bioimpedance-sensing electrode exits a distal boundary of the tumor.

For some applications, the bioimpedance-sensing electrode is disposed proximally to the electrode, and moving the distal region of the device through the tumor until the bioimpedance-sensing electrode exits the tumor includes moving the distal region of the device proximally through the tumor until the bioimpedance-sensing electrode exits a proximal boundary of the tumor.

For some applications:
the electrode is a first electrode, mounted on a first part of the distal region
the distal region further includes a second electrode, mounted on a second part of the distal region,
the first part and the second part are manipulable such that the first electrode is movable with respect to the second electrode, and
applying the electroporation pulse to the tumor using the electrode includes applying the electroporation pulse between the first electrode and the second electrode.

For some applications:
the first part is a distal part of a first shaft,
the second part is a distal part of a second shaft, and
positioning the electrode within the boundary of the tumor includes positioning the electrode within the boundary of the tumor by sliding the first shaft through the second shaft.

For some applications, sensing bioimpedance of the tissue includes sensing bioimpedance of the tissue between the first part and the second part.

For some applications, the method includes, subsequently to applying the electroporation pulse:
repositioning the electrode within the tumor, and
applying an other electroporation pulse to the tumor using the electrode.

For some applications, the method further includes, subsequently to repositioning the electrode within the tumor and prior to applying the other electroporation pulse, sensing bioimpedance of the tissue using the distal region of the device.

There is further provided, in accordance with some applications, apparatus for use with a tumor, the apparatus including a tumor-ablating device that has a distal region that includes a first part, including:
a first bioimpedance-sensing electrode;
a first electroporation electrode, mounted at a fixed position with respect to the first bioimpedance-sensing electrode.

For some applications, the distal region further includes a second part, including:
a second bioimpedance-sensing electrode; and
a second electroporation electrode, mounted at a fixed position with respect to the second bioimpedance-sensing electrode.

For some applications, the distal region is manipulable in a manner that changes an axial distance between the first part and the second part.

For some applications, the first part is a first part of a distal region of a first shaft of the device, and the second part is a second part of a distal region of a second shaft of the device.

For some applications, at the distal region, the first shaft and the second shaft fork with respect to each other.

For some applications, each of the first shaft and the second shaft are adapted to access the tumor from independent tumor-access sites.

For some applications, the distal region is manipulable in the manner that changes the axial distance between the first part and the second part by the first shaft being slidable through the second shaft.

For some applications, the first shaft is advanceable out of a distal end of the second shaft.

For some applications, the first bioimpedance-sensing electrode is disposed on the first shaft distally to the first electroporation electrode.

For some applications, the second bioimpedance-sensing electrode is disposed on the second shaft proximally from the second electroporation electrode.

For some applications, the first bioimpedance-sensing electrode is disposed on the first shaft proximally from the first electroporation electrode.

For some applications, the second shaft defines a side-port out of which the first shaft is advanceable.

For some applications, the first shaft is curvable with respect to the second shaft.

There is further provided, in accordance with some applications, an apparatus for ablating a tumor in a lung of a subject, the apparatus including a device that includes:
a shaft;
a bioimpedance-sensing electrode attached to a distal region of the shaft; and
an electroporation electrode, attached to the distal region of the shaft at a known distance from the bioimpedance-sensing electrode.

For some applications, the distal region of the shaft is advanceable into a position within the lung in which:
the electroporation electrode is disposed within a boundary of the tumor, and
the bioimpedance-sensing electrode is disposed outside of the boundary of the tumor.

There is further provided, in accordance with some applications, an apparatus for ablating a tumor, the apparatus including a device that includes:
a shaft;
a first bioimpedance-sensing electrode disposed on a distal region of the shaft;
a second bioimpedance-sensing electrode disposed on the distal region of the shaft; and
an electroporation electrode, disposed on the shaft axially between the first and second bioimpedance-sensing electrodes.

There is further provided, in accordance with some applications, a method for ablating a tumor, the method including:
advancing a distal region of a tumor-ablating device into the tumor, the distal region having an electroporation electrode mounted thereon, the electroporation electrode flanked by a proximal bioimpedance-sensing electrode and a distal bioimpedance-sensing electrode.

For some applications, the method further comprises determining a distal boundary of the tumor by sensing bioimpedance, facilitated by the distal bioimpedance-sensing electrode.

For some applications, the method further comprises, responsively to determining the distal boundary, and while the electroporation electrode is disposed within the distal boundary, driving the electroporation electrode to apply a first electroporation pulse to the tumor.

For some applications, the method further comprises determining a proximal boundary of the tumor by sensing bioimpedance, facilitated by the proximal bioimpedance-sensing electrode.

For some applications, the method further comprises, responsively to determining the proximal boundary, and while the electroporation electrode is disposed within the proximal boundary, driving the electroporation electrode to apply a second electroporation pulse to the tumor.

For some applications, driving the electroporation electrode to apply the first electroporation pulse to the tumor includes driving the electroporation electrode to apply the first electroporation pulse to the tumor prior to applying the second electroporation pulse to the tumor.

For some applications, driving the electroporation electrode to apply the first electroporation pulse to the tumor includes driving the electroporation electrode to apply the first electroporation pulse to the tumor subsequently to applying the second electroporation pulse to the tumor.

For some applications, the method further includes applying a plurality of electroporation pulses to the tumor, as the distal region of the device is advanced through the tumor.

There is further provided, in accordance with some applications, a method for use at a lung of a subject, the method including receiving information indicative of oscillation of bioimpedance of a tissue in the lung.

For some applications, the method further comprises responsively determining whether the tissue is tumor tissue.

For some applications, determining whether the tissue is tumor tissue includes:
determining whether a magnitude of the oscillation is less than a threshold magnitude, and
responsively to determining that the magnitude is less than the threshold, determining that the tissue is tumor tissue.

For some applications, determining whether the tissue is tumor tissue includes:
determining whether a magnitude of the oscillation is above a threshold magnitude, and
responsively to determining that the magnitude is above the threshold, determining that the tissue is not tumor tissue.

For some applications, determining whether the tissue is tumor tissue further includes determining whether a frequency of the oscillation matches a frequency of oscillation of the portion of the breathing cycle.

There is further provided, in accordance with some applications, a method for use at a lung of a subject, the method including receiving information indicative of bioimpedance of a tissue in the lung.

For some applications, the method further comprises responsively determining whether the tissue is cancerous.

There is further provided, in accordance with some applications, apparatus for use with a tumor, the apparatus including a tumor-ablating device that has a distal region that includes:
an inner shaft having an electroporation electrode mounted thereon; and
an outer shaft, axially slidable over the inner shaft in a manner that facilitates adjustment of an effective length of the electroporation electrode such that:
   distal advancement of the outer shaft over the inner shaft progressively decreases an effective length of the electroporation electrode by progressively covering the electroporation electrode, and
   proximal withdrawal of the outer shaft over the inner shaft progressively increases an effective length of the electroporation electrode by progressively exposing the electroporation electrode.

For some applications, the outer shaft is formed from an electrical insulator.

For some applications:
the electroporation electrode is configured to apply an electroporation pulse to the tumor, and
the apparatus further includes a control unit that includes a power generator, electrically connectable to the electroporation electrode, and adapted to drive the treatment applicator to apply the electroporation pulse.

For some applications, the device includes a bioimpedance-sensing electrode mounted on the distal region.

For some applications:
the bioimpedance-sensing electrode is a first bioimpedance-sensing electrode,
the first bioimpedance-sensing electrode is mounted on the inner shaft, distally to the electroporation electrode, and
a second bioimpedance-sensing electrode is mounted on the outer shaft.

For some applications, the apparatus includes a control unit, adapted to receive a signal from the bioimpedance-sensing electrode, and to responsively provide an output indicative of bioimpedance of tissue adjacent the bioimpedance-sensing electrode.

For some applications, the control unit is adapted to identify a change in the signal over at least a portion of a breathing cycle of the patient, the output being indicative of a change in bioimpedance of the tissue adjacent the bioimpedance-sensing electrode over at least the portion of the breathing cycle.

For some applications, the control unit is configured to, responsively to the signal, output information indicative of the position of the bioimpedance-sensing electrode with respect to the tumor.

For some applications:
the device includes a terminal, electrically connected to the bioimpedance-sensing electrode via a conductor extending along the shaft, and
the control unit is electrically connectable to the bioimpedance-sensing electrode by electrically connecting the control unit to the terminal.

There is further provided, in accordance with some applications, a method for ablating a tumor, the method including:
advancing a distal region of a tumor-ablating device into the tumor, the device including:
   an inner shaft having an electroporation electrode mounted thereon, and
   an outer shaft adapted to slide over the inner shaft.

For some applications, the method further comprises positioning a distal end of the electroporation electrode within the tumor.

For some applications, the method further comprises adjusting an effective length of the electroporation electrode by sliding the outer shaft over the inner shaft.

For some applications, the method further comprises driving the electroporation electrode to apply an electroporation pulse to the tumor.

For some applications, adjusting the effective length of the electroporation electrode by sliding the outer shaft over the inner shaft includes adjusting the effective length of the electroporation electrode until the electroporation electrode spans a desired length of the tumor.

For some applications, adjusting the effective length of the electroporation electrode until the electroporation electrode spans a desired length of the tumor includes adjusting the effective length of the electroporation electrode until the electroporation electrode spans an entire length of the tumor.

For some applications:
the distal region further includes a bioimpedance-sensing electrode, and
adjusting the effective length of the electroporation electrode includes adjusting the effective length guided by sensing performed by the bioimpedance-sensing electrode.

For some applications, adjusting the effective length guided by sensing performed by the bioimpedance-sensing electrode includes moving the distal region of the device through the tumor until the bioimpedance-sensing electrode exits the tumor.

For some applications, adjusting the effective length guided by sensing performed by the bioimpedance-sensing electrode includes adjusting the effective length guided by sensing performed by the bioimpedance-sensing electrode while the bioimpedance-sensing electrode is electrically connected to a control unit, the control unit being configured to provide an alert in response to a change in bioimpedance detected via the bioimpedance-sensing electrode upon the exit of the bioimpedance-sensing electrode from the tumor.

For some applications, the bioimpedance-sensing electrode is disposed distally to the electroporation electrode on the inner shaft, and moving the distal region of the device through the tumor until the bioimpedance-sensing electrode exits the tumor includes moving the inner shaft distally through the tumor until the bioimpedance-sensing electrode exits a distal boundary of the tumor.

For some applications:
the bioimpedance-sensing electrode is a first bioimpedance-sensing electrode, and
the distal region further includes a second bioimpedance-sensing electrode, disposed on the outer shaft, and
moving the distal region of the device through the tumor until the bioimpedance-sensing electrode exits the tumor includes moving the outer shaft proximally through the tumor until the second bioimpedance-sensing electrode exits a proximal boundary of the tumor.

The present invention will be more fully understood from the following detailed description of applications thereof, taken together with the drawings, in which:

### BRIEF DESCRIPTION OF THE DRAWINGS

FIG. 1 is a schematic illustration of a device for electroporation ablation, in accordance with some embodiments of this disclosure;

FIG. 2 is a schematic illustration of a device for electroporation ablation guided by bioimpedance-sensing, in accordance with some embodiments of this disclosure;

FIG. 3 is a schematic illustration of a device for electroporation ablation, in accordance with some embodiments of this disclosure;

FIG. 4 is a cross sectional view of a shaft, in accordance with some embodiments of this disclosure;

FIG. 5 is a schematic illustration of a device for electroporation ablation, in accordance with some embodiments of this disclosure;

FIGS. 6A-D are a schematic illustration of at least some steps in a technique for ablating a tumor, guided by bioimpedance sensing, in accordance with some embodiments of this disclosure;

FIGS. 7-8, and 9A-B are schematic illustrations of devices for electroporation ablation comprising multiple distal regions, in accordance with some embodiments of this disclosure;

FIGS. 10 and 11A-C are schematic illustrations of a device for electroporation ablation guided by bioimpedance sensing, in accordance with some embodiments of this disclosure; and

FIG. 12 is a schematic illustration of a device for electroporation ablation guided by bioimpedance sensing, in accordance with some embodiments of this disclosure; and

FIGS. 13A-C are schematic illustrations of a device for electroporation ablation guided by bioimpedance sensing, in accordance with some embodiments of this disclosure.

### DETAILED DESCRIPTION

Methods and systems are provided for determining the boundaries of a tumor using electrical impedance, in order to position a treatment applicator within the boundaries of the tumor. For example, the treatment applicator may comprise treatment electrodes, such as electroporation electrodes. It may be advantageous to position the treatment electrodes within the boundaries of the tumor, e.g. to focus the treatment on tumor tissue rather than surrounding tissue. It may further be advantageous to position the treatment electrodes close to the boundaries of the tumor in order to treat the entire tumor—e.g. in order to avoid leaving tumor cells near the boundaries of the tumor untreated.

For some applications, due to differences in tissue properties between the tumor itself and its surrounding tissue, it may be possible to determine the boundaries of the tumor by sensing bioimpedance. For example, healthy lung tissue typically has high bioimpedance (e.g. due to the air-filled structures of the parenchyma and its alveoli), whereas a lung tumor typically has substantially lower bioimpedance. Thus, bioimpedance-sensing electrodes positioned close to, and/or within, the tumor may be able to provide information regarding the boundaries of a tumor—particularly a lung tumor. Furthermore, this difference in bioimpedance between the tumor and its surrounding tissue may allow for successful electroporation of the tumor, while minimizing collateral damage to the surrounding tissue. For example, the relatively high conductivity of the tumor, relative to the surrounding tissue, may facilitate conduction of an electroporation pulse throughout the tumor but not into the surrounding tissue.

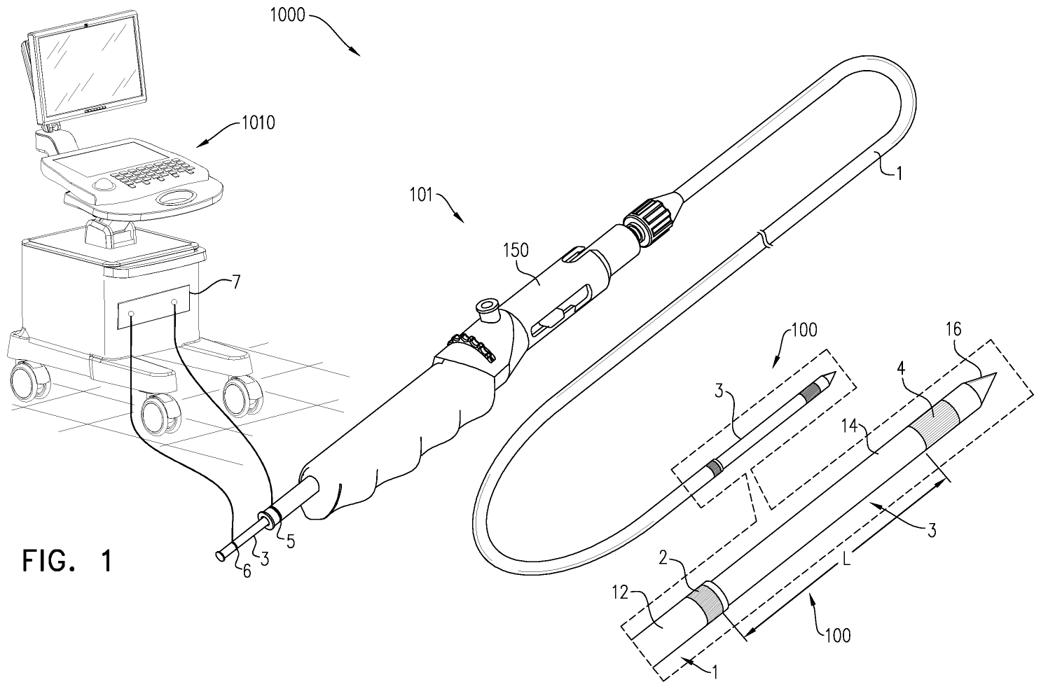

Reference is now made to FIG. 1, which is a schematic illustration of a system 1000 for electroporation ablation of a tumor, in accordance with some applications. System 1000 comprises a device (e.g. an apparatus) 101, and may further comprise a control unit 1010. Control unit 1010 may comprise a power generator (or power source) 7.

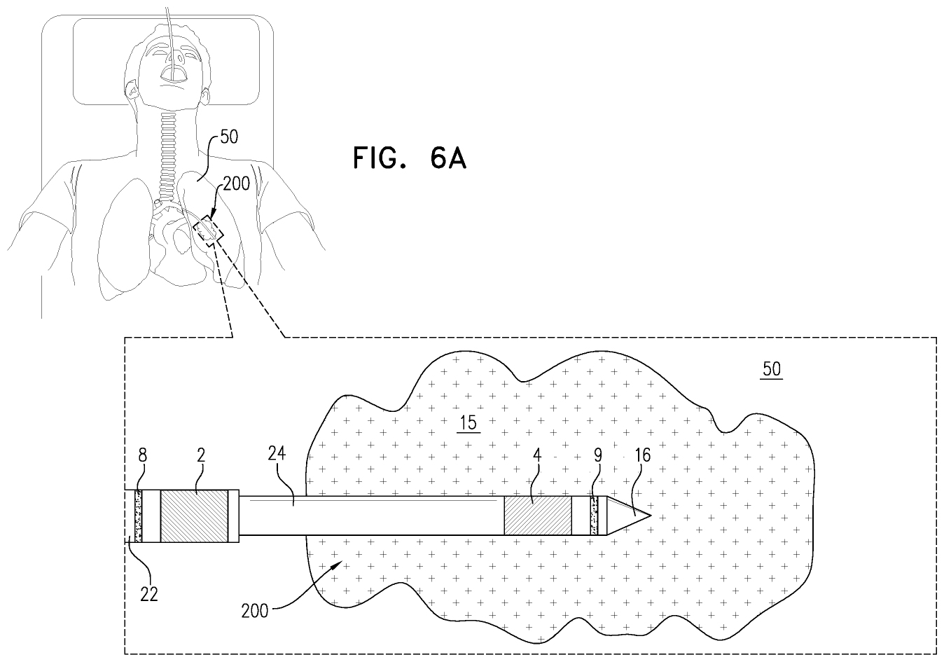

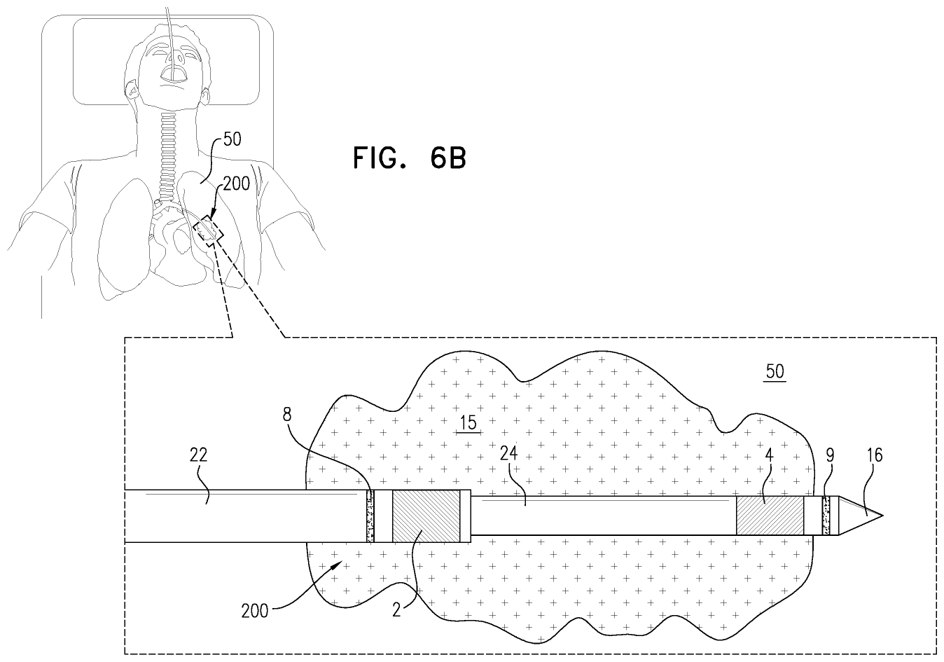

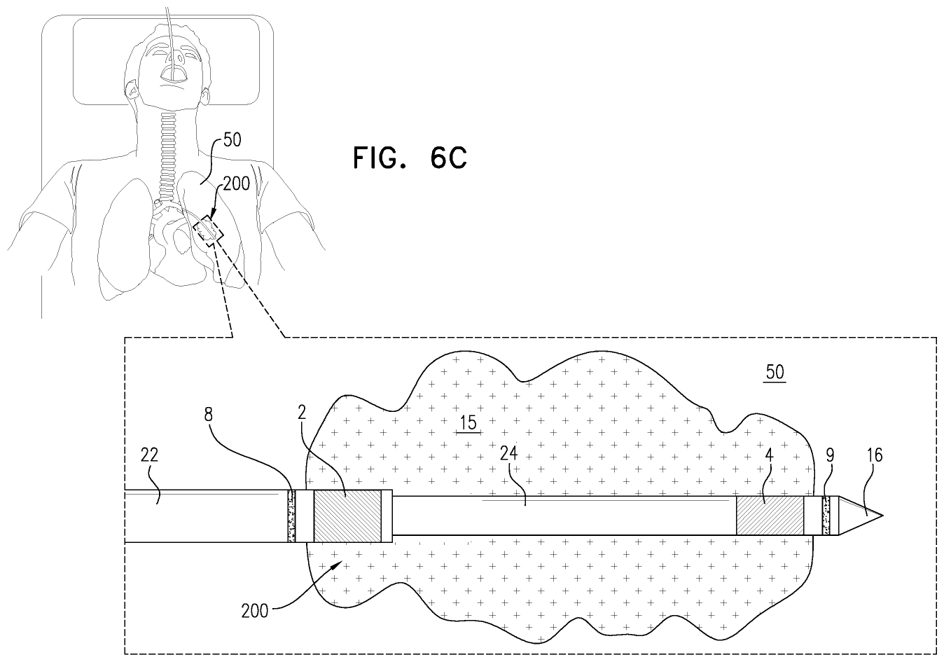

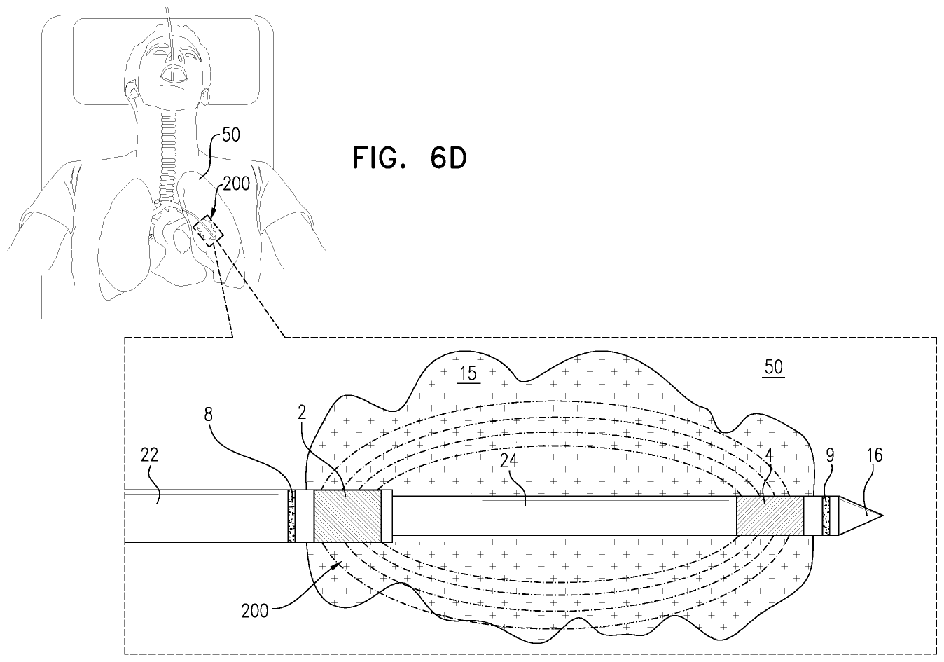

As illustrated in FIG. 1, device 101 may comprise at least one shaft (e.g. a tube, catheter, rod, and/or bronchoscope), such as a shaft 1, and has a distal region 100. At least part of distal region 100 is adapted to be advanced into a tumor. For some applications, distal region 100 may access the tumor by penetrating a body lumen through which they are guided. For some applications, device 101 is advanced into a patient, e.g. into a lung 50 of a patient, as illustrated in FIGS. 6A-D. For applications in which the tumor is a lung tumor, the apparatus may be advanced into the patient transbronchially (e.g. via a bronchoscope)—e.g. via the nose or mouth of the patient, down the trachea, and into an airway (e.g. a bronchus) of the patient. For some such applications, distal region 100 may then penetrate the airway (e.g. using a tissue-piercing tip 16 of the distal region) and enter the parenchyma adjacent the tumor. For some such applications, shaft 1 may be flexible. For some applications, the advancement is performed percutaneously (e.g. transluminally or transthoracically). For some such applications, shaft 1 may be rigid.

A first electroporation electrode 4 is disposed at distal region 100 (e.g. the electrode may be attached to a shaft of the device) such that advancing the distal region into the tumor also advances electrode 4 into the tumor. For some embodiments, a second electroporation electrode 2 may also be disposed at distal region 100—e.g. such that it, too, becomes positioned within the tumor, such that an electroporation pulse can be applied between the first electrode and the second electrode in order to electroporate the tumor. For some applications, electrodes 2 and 4 can be considered to be components of (e.g. may collectively define) a treatment applicator of device 101.

Distal region 100 is reversibly lengthenable in a manner that changes an axial distance L between electrodes 2 and 4. For some applications, distal region 100 may include a telescopic assembly—e.g. having a distal portion 14 and a proximal portion 12 which are axially slidable with respect to each other. For some such applications, shaft 1 is an outer shaft, through which an inner shaft 3 of device 101 is axially slidable. For some such applications, first electrode 4 is disposed on distal portion 14 of the distal region, and second electrode 2 is disposed on proximal portion 12 of the distal region. Distal portion 14 may be defined by a distal part of shaft 3. Proximal portion 12 may be defined by a distal part of shaft 1.

For some such applications, shafts 1 and 3 are coaxial. As described hereinabove, it may be advantageous to position electrodes 2 and 4 within the tumor, close to (e.g. at) respective (e.g. opposing) boundaries of the tumor, in order to successfully electroporate the entire tumor. For such applications, such positioning of electrodes 2 and 4 tumor can be facilitated by lengthening and/or shortening (e.g. telescopically) in order to adjust the distance L between the electrodes.

Control unit 1010 (e.g. power generator 7 thereof) is adapted to drive electrodes 2 and 4 to apply the electroporation pulse therebetween. At a proximal region 150 of device 101, the device may comprise terminals 5 and 6, via which electrodes 2 and 4, respectively, may be electrically connected to control unit 1010 (e.g. to power generator 7). For example, wires extending from the control unit may be connected to the terminals—e.g. prior to use. Electrodes 2 and 4 may be electrically connected to terminals 5 and 6, respectively, via conductors (e.g. wires) that extend along the corresponding shaft(s). For example, one conductor may extend from electrode 4, along shaft 3 (e.g. via a lumen thereof, and/or within a side-wall thereof) to proximal terminal 6, and/or another conductor may extend from electrode 2, along shaft 1 (e.g. via a lumen thereof, and/or within a side-wall thereof) to proximal terminal 5. The wires may be electrically insulated from each other.

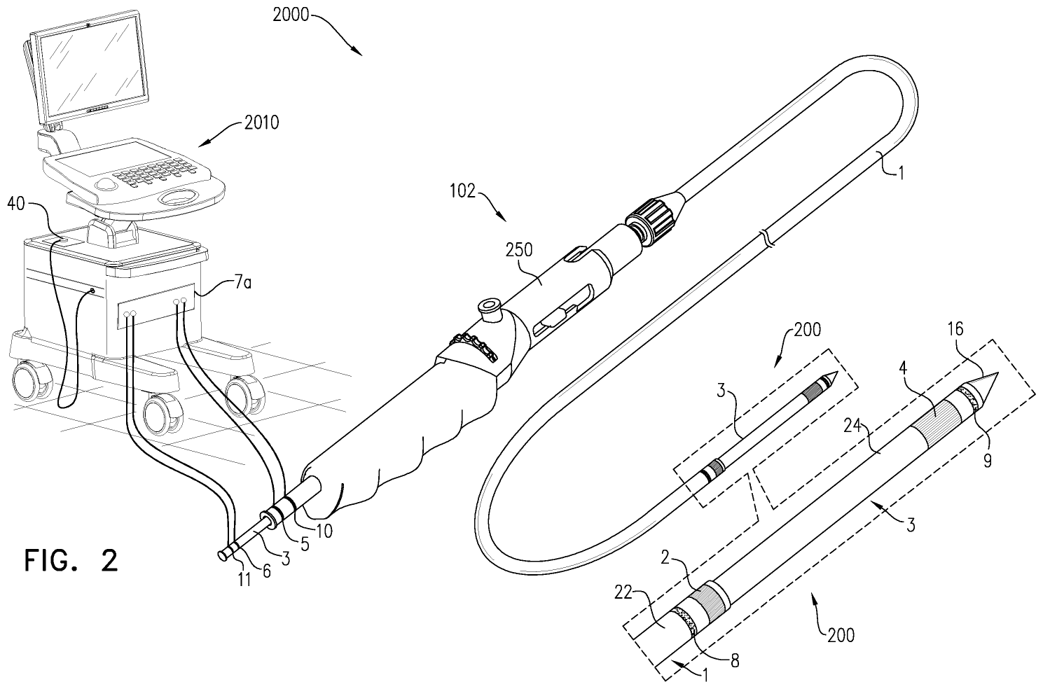

Reference is now made to FIGS. 2 and 6A-D, which are a schematic illustration of a system 2000 for electroporation ablation of a tumor 15, in accordance with some applications. System 2000 comprises a device (e.g. an apparatus) 102, and may further comprise a control unit 2010. Control unit 2010 may comprise a power generator (or power source) 7a. Similarly to device 101, device 102 may comprise at least one shaft 1 (e.g. a flexible tube, catheter, rod, and/or bronchoscope), and has a distal region 200, adapted to be advanced into tumor 15. Also similarly to device 101, a first electroporation electrode 4 and a second electroporation electrode 2 are disposed at the distal region 200, in order to apply an electroporation pulse to ablate a tumor. For some applications, distal region 200 is reversibly lengthenable in a manner that changes an axial distance between electrodes 2 and 4, e.g. via distal region 200 including a telescopic assembly (e.g. by a telescopic arrangement of shafts 1 and 3), or by any other means. For some applications, system 2000 may be considered to be a variant of system 1000, e.g. device 102 may be considered to be a variant of device 101.

Device 102 is similar to device 101 mentioned above, but may also comprise, at its distal region 200, one or more bioimpedance-sensing electrodes, such as a first bioimpedance-sensing electrode 8 and a second bioimpedance-sensing electrode 9. For some applications, these bioimpedance-sensing electrodes are discrete electrodes that are distinct from electroporation electrodes 2 and 4, and may be electrically connected to control unit 2010 independently from the electroporation electrodes.

Although each of the bioimpedance-sensing electrodes described herein is illustrated and referred to as a single electrode, any of these bioimpedance-sensing electrodes may, in fact, represent a set of electrodes that may cooperate—e.g. to serve as a bioimpedance sensor (e.g. further in cooperation with control unit 2010). That is, for some applications, any of the bioimpedance-sensing electrodes described herein can be comprised of more than one (e.g. multiple) electrodes. For some such applications, such a bioimpedance-sensing electrode (comprising multiple individual electrodes) may be considered a bioimpedance sensor, either alone, or in combination with control unit 2010.

As well as being adapted to drive electrodes 2 and 4 to apply the electroporation pulse therebetween (e.g. via power generator 7a), control unit 2010 may be adapted to receive signals from bioimpedance sensing electrodes 8 and 9 in order to determine the positions of electrodes 2 and 4 with respect to the tumor, as described hereinbelow. At a proximal region 250 of device 102, the device may comprise terminals 5 and 6, via which electrodes 2 and 4, respectively may be electrically connected to control unit 2010 (e.g. to power generator 7)—e.g. as described for device 101. In addition, device 102 typically further comprises terminals 10 and 11, via which electrodes 8 and 9 respectively may be electrically connected to the control unit. For example, and as shown, wires extending from the control unit may be connected to the terminals. The wires may be electrically insulated from each other.

For some applications, the bioimpedance sensing may be performed by the bioimpedance-sensing electrode(s) applying an electrical current to the tissue (e.g. between the two electrodes), and measuring the resulting current. For some applications, control unit 2010 is adapted to drive the bioimpedance sensing, e.g. by providing the electrical current which is used to perform the bioimpedance sensing. For some applications, power generator 7a is used to provide this electrical current, e.g. via terminals 10 and 11.

For some applications, bioimpedance sensing is performed in a potentiostatic mode. For some applications, bioimpedance sensing is performed in a galvanostatic mode. For some applications, the electrical current used to perform the bioimpedance sensing is a low voltage (e.g. at least 0.5 V and/or no more than 5 V) current. For some applications, the electrical current used to perform the bioimpedance sensing has a sinusoidal waveform. For some applications, the electrical current used to perform the bioimpedance sensing has a frequency of at least 10 Hz and/or no more than 1 MHZ. For some applications, the electrical current used to perform the bioimpedance sensing has an amplitude of at least 1 microampere and/or no more than 15 milliampere.

Once distal region 200 has been advanced to tumor 15, the boundaries of the tumor can be determined using bioimpedance-sensing electrodes 8 and 9. For example, due to the differences in tissue properties between the tumor and its surrounding tissue e.g. as described hereinabove, the device (e.g. control unit 2010 thereof), can determine (or can facilitate determination of) the boundaries of the tumor by moving (or facilitating movement of) bioimpedance-sensing electrodes 8 and 9 with respect to the tumor, and sensing the bioimpedance in the different positions. For example, and as schematically illustrated in FIGS. 6A-D, the bioimpedance-sensing electrodes may be advanced into and/or out of the tumor so that the precise position of the transition between inside and outside (i.e. the boundary) can be identified—e.g. by determining whether each of bioimpedance-sensing electrodes 8 and 9 is disposed inside or outside of the tumor.

For some applications, this determination is achieved by sensing bioimpedance between bioimpedance sensing electrodes 8 and 9 (e.g. the bioimpedance-sensing electrodes are used in a bipolar manner). For some applications, a remote electrode 40, such as a skin electrode or a grounding pad, is used. For example, the determination of whether bioimpedance-sensing electrode 8 is inside or outside of tumor 15 may be facilitated (or made) by sensing bioimpedance between bioimpedance-sensing electrode 8 and remote electrode 40, and a similar determination may be made for bioimpedance-sensing electrode 9 (e.g. each of bioimpedance-sensing electrodes 8 and 9 is used in a monopolar manner), such that control unit 2010 can verify the position of each of the bioimpedance-sensing electrodes independently of the other. For some applications, a combination of bipolar and unipolar sensing is performed. For some applications, electrode 8 or electrode 9 may be replaced with a skin electrode or a grounding pad.

For some applications, the bioimpedance between each bioimpedance-sensing electrode and its respective electroporation electrode may be determined. For example, the bioimpedance between bioimpedance-sensing electrode 8 and electroporation electrode 2 may be detected in order to determine whether electroporation electrode 2 is within the boundary of the tumor. Additionally or alternatively, the bioimpedance between bioimpedance-sensing element 9 and electroporation electrode 4 may be detected in order to determine whether electroporation electrode 4 is within the boundary of the tumor.

For some applications, bioimpedance sensing is performed between a combination of bioimpedance-sensing electrodes, electroporation electrodes, and/or remote electrodes, for example, using two, three, or more (e.g. four) of electrodes 2, 4, 8, 9, and 40 to perform the bioimpedance sensing. For some applications, more than one sensing electrode may be used proximal and/or distal to the electroporation electrodes. For example, there may be a pair of bioimpedance-sensing electrodes positioned proximal to electrode 2 and/or a pair of bioimpedance sensing electrodes positioned distal to electrode 2, and bioimpedance may be sensed between the electrodes of the proximal pair and/or the electrodes of the distal pair. Similarly, there may be a pair of bioimpedance-sensing electrodes positioned proximal to electrode 4 and/or a pair of bioimpedance sensing electrodes positioned distal to electrode 4, and bioimpedance may be sensed between the electrodes of the proximal pair and/or the electrodes of the distal pair. In some embodiments bioimpedance is measured between a distalmost bioimpedance-sensing electrode and a proximal most bioimpedance-sensing electrode.

For some applications, a remote electrode (such as remote electrode 40 or similar, e.g. a skin electrode), may be used to facilitate delivery of an electroporation pulse to the tumor—e.g. with the remote electrode serving as a return electrode. For example, the pulse may be delivered via electrode 4, electrode 2, or both electrodes 4 and 2 cooperating to serve as a single electrode. For example, electrode 2 or electrode 4 may be replaced with a skin electrode or a grounding pad. This may be considered a “monopolar” configuration, whereas application of the pulse between electrodes 2 and 4 may be considered a “bipolar” configuration. For some applications, a single tumor may be treated by a combination of both monopolar and bipolar pulses.

For some applications, control unit 2010 is adapted to provide an output e.g. a visual output such as via a screen, and/or an audible output and/or a tactile or haptic output, responsively to bioimpedance sensing performed by the bioimpedance-sensing electrode(s). For example, a representation of the boundaries of the tumor (e.g. a map showing the boundaries of the tumor) may be outputted by the control unit, such that an operator can position the electroporation electrode(s) within the boundaries. For some applications, the output is discrete e.g. a textual output representing the tissue type in which the bioimpedance-sensing electrode (and/or the electroporation electrode) is determined to be disposed.

Device 102 is configured such that the position of its treatment applicator (e.g. electrodes 2 and 4) is in a fixed and/or known position with respect to its bioimpedance-sensing electrodes 8 and 9, such that positioning of electrodes 8 and 9 just outside of the boundaries of the tumor automatically positions (or facilitates positioning of) the treatment applicator just within the boundaries of the tumor. For example, and as illustrated in FIGS. 2, and 6A-D, due to the arrangement of having electroporation electrodes 2 and 4 being positioned axially between bioimpedance-sensing electrodes 8 and 9, verifying that the bioimpedance sensing electrodes are at (e.g. just outside of) the boundaries of tumor 15 may automatically verify that electroporation electrodes 2 and 4 are just within the boundaries—e.g. no more than 5 mm (e.g. no more than 4 mm, e.g. no more than 3 mm, such as no more than 2 mm) and/or at least 0.5 mm (e.g. at least 1 mm, e.g. at least 2 mm, such as at least 3 mm) within the boundaries, such as 0.5-5 mm within the boundaries, of the tumor. For some applications, rather than positioning electrodes 8 and 9 just outside of the boundaries of tumor 15, a similar technique may be used in which they are positioned just inside of the boundaries.

For some applications, and as shown, distal region 200 may include a telescopic assembly—e.g. having a distal portion 24 (which may be a distal part of shaft 3) and a proximal portion 22 (which may be a distal part of shaft 1) which are axially slidable with respect to each other. For some applications, shafts 1 and 3 are coaxial. For some such applications, electroporation electrode 4 is disposed on distal portion 24, with bioimpedance-sensing electrode 9 disposed on the distal portion just distally—e.g. greater than 0.5 mm (e.g. greater than 1 mm) and/or less than 10 mm (e.g. less than 5 mm) distally—from electroporation electrode 4, such that positioning bioimpedance-sensing electrode 9 just beyond the boundary of the tumor positions electroporation electrode 4 just within the boundary. That is, electrode 4 becomes positioned on an opposite sides of the boundary to electrode 9, with the boundary between the electrodes. Thus, bioimpedance-sensing electrode 9 can be used by control unit 2010 to detect the distal boundary and provide an alert (e.g. a visual, audible, tactile and/or haptic alert) indicating, for example, that no further distal advancement is required (or desired). Similarly, second electrode 2 is disposed on proximal portion 22, with bioimpedance-sensing electrode 8 disposed on the proximal portion just proximally—e.g. greater than 0.5 mm (e.g. greater than 1 mm) and/or less than 5 mm (e.g. less than 10 mm) proximally—from electroporation electrode 2, such that positioning bioimpedance-sensing electrode 8 just before (e.g. proximally from) the boundary of the tumor positions electroporation electrode 2 just within the boundary.

FIGS. 6A-D may represent a series of steps that may be performed by the operator, to position electroporation electrodes 2 and 4 at opposite boundaries of tumor 15. These steps are typically facilitated by bioimpedance-sensing electrodes 8 and 9 indicating the position of the electroporation electrodes 2 and 4 with respect to tumor 15, as will be described hereinbelow. It is to be noted that FIGS. 6A-D are primarily intended to illustrate the capability of system 2000, rather than to strictly define a sequence of steps of a procedure.

Distal region 200 is initially advanced into tumor 15 such that bioimpedance-sensing electrode 9 and electroporation electrode 4 enter the tumor (FIG. 6A). The fact that electrode 9 is disposed within the tumor is identifiable by bioimpedance sensing.

Distal region 200 continues to be advanced through tumor 15, until bioimpedance-sensing electrode 9 exits the tumor—e.g. the distal boundary of the tumor (FIG. 6B). For example, control unit 2010 may provide an alert to the operator in response to a change in bioimpedance detected via electrode 9 upon its exit from tumor 15. As described hereinabove, this positioning of bioimpedance-sensing electrode 9 just outside of the boundary of the tumor positions electroporation electrode 4 just within the boundary. In the particular example shown, distal region 200 is advanced into tumor 15 while the distance between electrodes 4 and 2 (i.e. distance L, described hereinabove) is sufficiently small that positioning of electrode 4 just inside the distal boundary of the tumor positions both electroporation electrode 2 and bioimpedance-sensing electrode 8 within the tumor (FIG. 6B). However, it is to be understood that this is dependent on the particular application of device 102 and the dimensions of the particular tumor.

FIG. 6C illustrates electrodes 2 and 8 being moved proximally by axially extending the length of distal region 200 (e.g. telescopically withdrawing proximal portion 22 of the distal region from distal portion 24) to position electrode 2 just within a proximal boundary of the tumor. Similarly to that described with reference to distal electrode 9, control unit 2010 may provide an alert to the operator in response to a change in bioimpedance detected via electrode 8 upon its exit from tumor 15. As described hereinabove, this positioning of bioimpedance-sensing electrode 8 just outside of the boundary of the tumor positions electroporation electrode 2 just within the boundary.

In this position, an electroporation pulse is then be applied to the tumor, as represented by the field lines in FIG. 6D.

For some applications, control unit 2010 is adapted to adjust the electroporation pulse applied by power generator 7a, responsively to the bioimpedance sensing. For example, control unit 2010 may be adapted to monitor the treatment progress, by monitoring changes in the tumor's impedance using bioimpedance-sensing electrodes 8 and 9. For some applications, this monitoring is performed iteratively and/or continuously throughout the ablation process.

For some applications, bioimpedance sensing is further performed during and/or after the ablation process to optimize the result.

For some applications, the electroporation pulse is applied whilst the bioimpedance-sensing electrode(s) are just within the boundary of the tumor, rather than being disposed just outside the boundary.

For some applications, in order to ensure full coverage of the tumor, following the application of an initial electroporation pulse to tumor 15, electrodes 2 and 4 may be moved within the tumor and a second electroporation pulse may be applied. For example, the electrode 4 may be pulled back towards the electrode 2, e.g. by telescopically shortening distal region 200 by pulling distal portion 24 proximally. Alternatively or additionally, electrode 2 may be pushed forward towards electrode 4, e.g. by pushing proximal region 22 distally. In some embodiments, after initial electroporation pulsing, both electrodes may be removed and re-inserted at a different angle or from a different entry point. In some embodiments, one electrode may be left in place and the other electrode inserted at a different angle.

In some embodiments, multiple shafts (e.g. multiple instances of shaft 3), each having an electroporation electrode thereon may be passed through shaft 1 (e.g. simultaneously). For some applications, each shaft 3 has a bioimpedance-sensing electrode mounted thereon, to provide information regarding its respective electroporation electrode with respect to the boundary of the tumor, e.g. as described hereinabove with reference to FIGS. 2 and 6A-D. For some applications, each of these shafts 3 are directed at a different angle within the tumor, in order to ensure complete coverage.

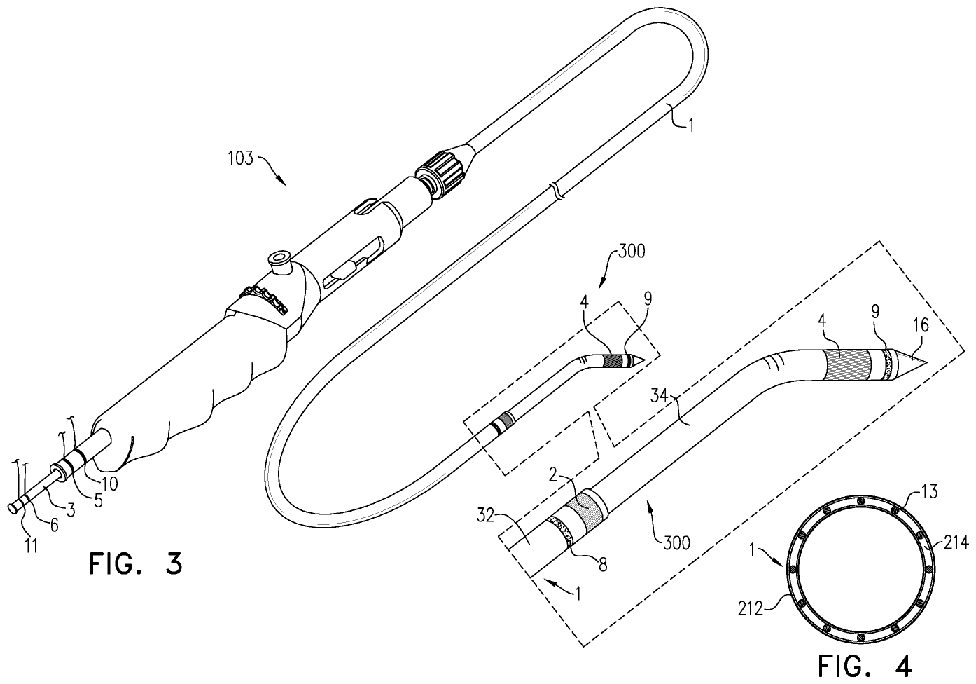

Reference is now may to FIG. 3, which is a schematic illustration of a device 103, in accordance with some applications. Device 103 may be considered to be a variant of device 101 and/or device 102, for example, device 103 may be a part of a system such as system 2000, and/or can be compatible with a control unit, such as control unit 2010. Similarly to the devices mentioned hereinabove, device 103 has a distal region 300, adapted to be advanced into a tumor. Distal region 300 defines a proximal portion 32 and a distal portion 34, the distal portion being telescopically extendable with respect to the proximal portion. Similarly to the devices described above, a first electroporation electrode 4 is disposed on the distal portion, and a second electroporation electrode 2 is disposed on the proximal portion. Bioimpedance sensing electrodes may be disposed on each of the distal portion and the proximal portion respectively, as described with reference to device 102 hereinabove.

As illustrated in FIG. 3, distal portion 34 is curved (or is actively bendable) with respect to proximal portion 32, such that electrode 4 can be oriented within the tumor at an angle to electrode 2. This may advantageously allow for positioning of electrodes 2 and 4 at opposite or suitably distanced boundaries of the tumor, to allow for complete eradication of the tumor.

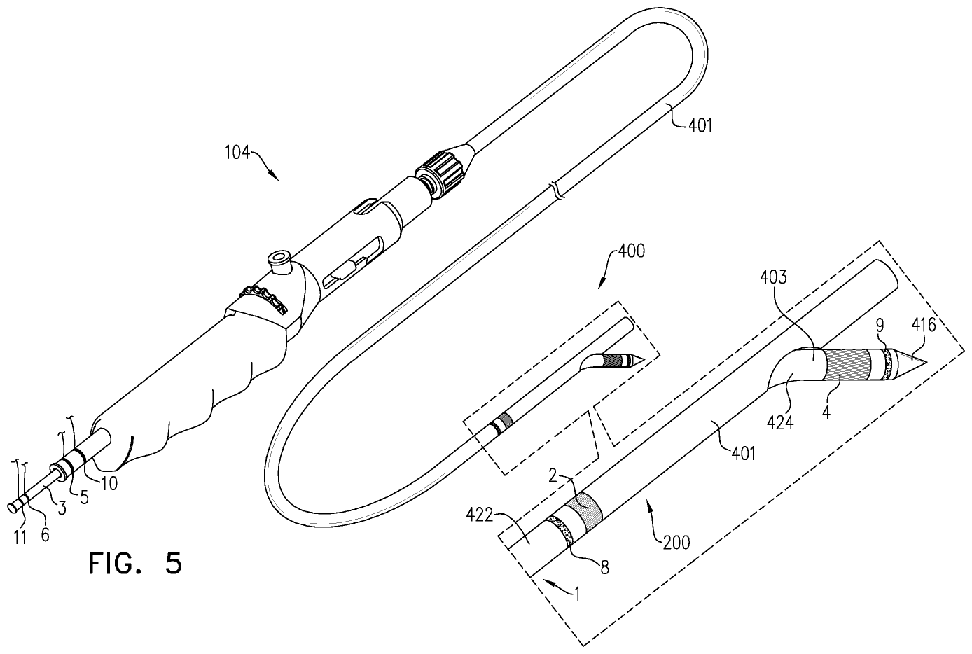

Reference is now made to FIG. 5, which is a schematic illustration of a device 104 for tumor ablation in accordance with some embodiments of the present disclosure. Device 104 may be considered to be a variant of device 101 and/or device 102 and/or 103, having a distal region 400, adapted to be advanced into a tumor. Distal region 400 defines a proximal portion 422 and a distal portion 424, the distal portion being telescopically extendable with respect to the proximal portion. Similarly to the devices described above, a first electroporation electrode 404 is disposed on the distal portion, and a second electroporation electrode 402 is disposed on the proximal portion. Bioimpedance sensing electrodes may be disposed on each of the distal portion and the proximal portion respectively, as described with reference to device 102 hereinabove. Also similarly to the devices mentioned above, device 104 comprises a shaft 401, through which a shaft 403 is passable, such that at distal region 400, shaft 403 forms the distal portion 424, and shaft 401 forms proximal portion 422.

Device 104 can be similar to the devices mentioned above, but shaft 401 may define a side port through which shaft 403 can be passed, such that electrode 404 can be positioned at selected boundaries of the tumor. The distal end of hollow shaft 401 may either be closed or open. In some embodiments shaft 401 includes more than one side port through which either multiple shafts 403 may be passed, or the same shaft 403 passed in a selective manner, such that the electrode 404 can be positioned at various boundaries of the tumor. This may advantageously allow for positioning of electrodes 402 and 404 at opposite or suitably distanced boundaries of the tumor, to allow for complete eradication of the tumor.

Reference is now made to FIGS. 7, 8, and 9A-B, which are schematic illustrations of devices that facilitate placement of more than one distal region (e.g. more than one treatment applicator) within a tumor, in accordance with some applications.

For some applications, a device having more than one distal region is advanced into the tumor, each distal region of the device having at least one treatment applicator and/or electroporation electrode disposed thereon. For some applications, each distal region also has a bioimpedance-sensing electrode disposed thereon, e.g. to determine whether the electroporation electrode on that distal region is located within, but at a boundary of, the tumor. Having more than one distal region may advantageously allow for the positioning of the electroporation electrodes at various boundaries of the tumor, in order to ensure complete coverage of the tumor. For example, FIGS. 7-9B can be understood as such devices having more than one distal region for positioning electroporation electrodes.

FIG. 7 shows a system 7000 comprising multiple devices 102 being advanced into a tumor 15, with each device having at least one electroporation electrode disposed thereon. Each of devices 102 may be considered to be a variant of, or substantially identical to, device 102 as described with reference to FIGS. 2 and 6A-D.

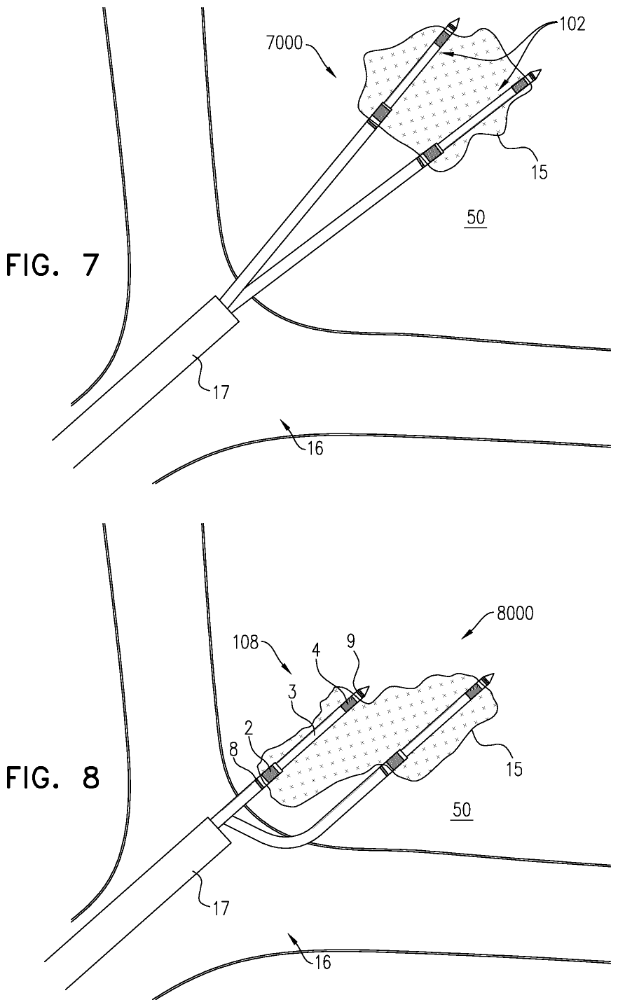

FIG. 8 shows a system 8000 comprising a device 108. System 8000 may be otherwise identical to system 7000 except that device 108 may fork into two separate distal regions, as shown. This may allow for accessing of the tumor via different access or entry points, such that the electrodes can advantageously be placed at various opposing boundaries of the tumor.

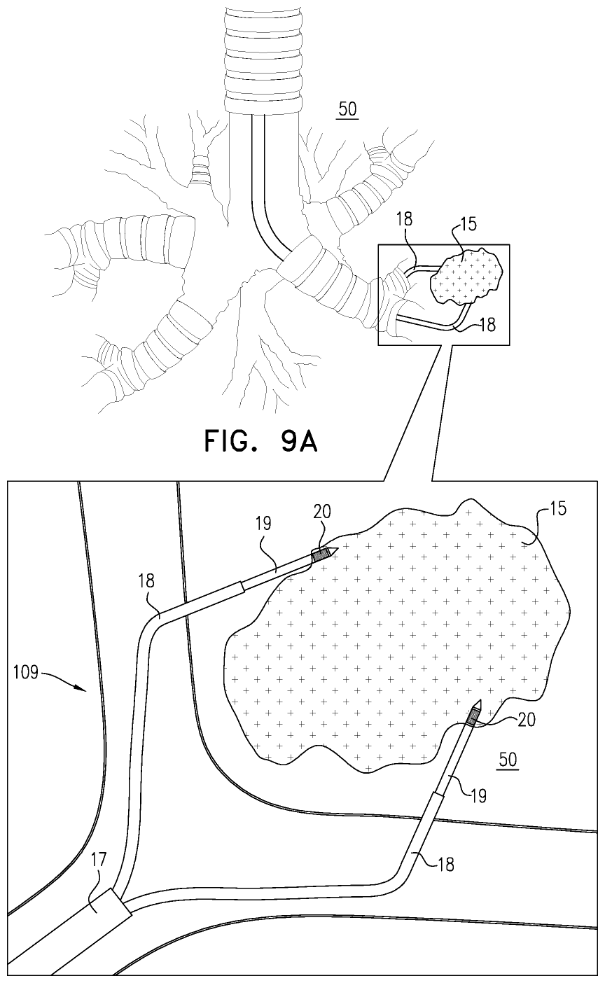

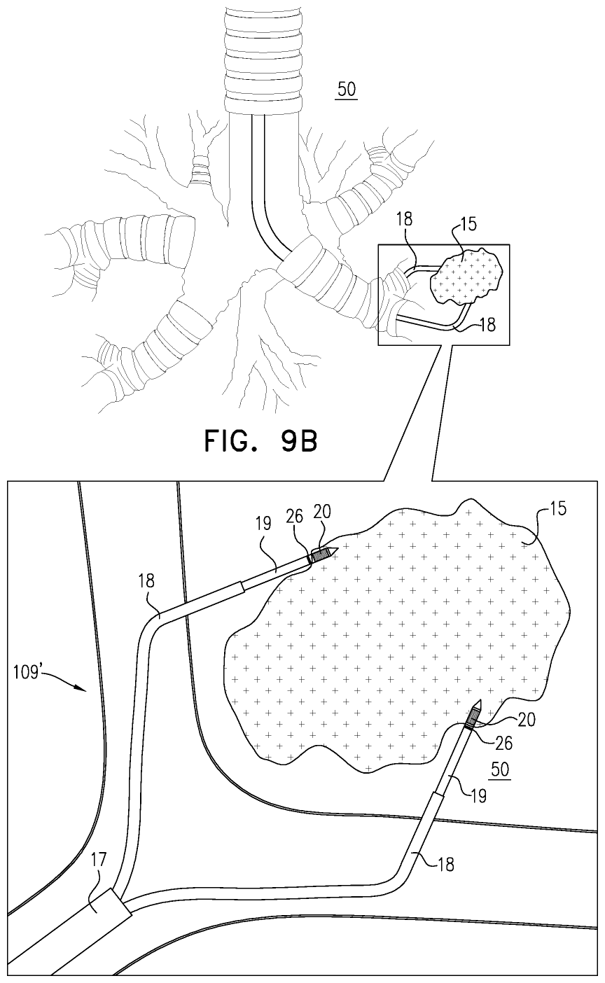

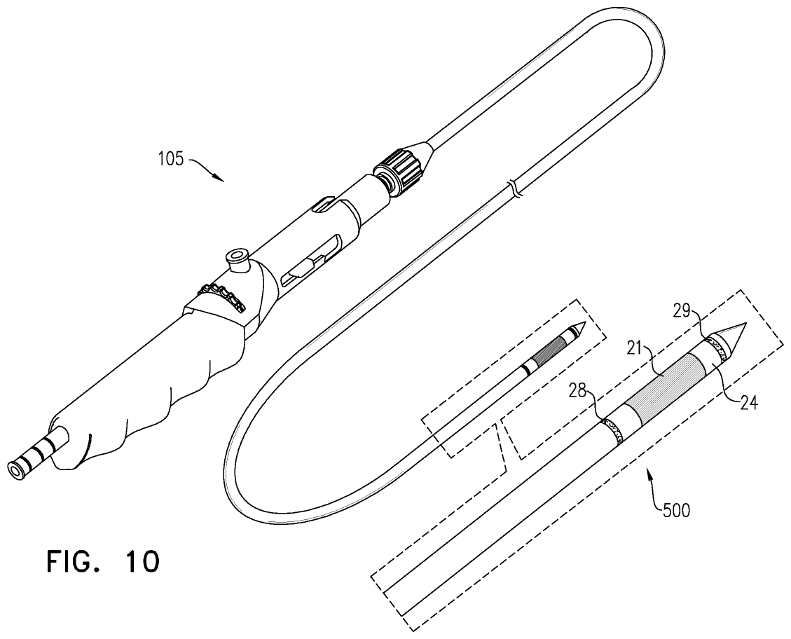

FIG. 9A illustrates a device 109 having a sheath (e.g. a bronchoscope) 17, out of which one or more shafts (e.g. steerable catheters) 18 may be advanced in order to direct distal regions 19, each having electroporation electrodes 20 disposed thereon, into a tumor 15 from various access points or specific angles or interest. Each steerable catheter 18 may be advanced into the tumor from different airways (e.g. bronchi), such that opposite and or distanced boundaries of the tumor are accessed by each electrode 20. For some applications, bioimpedance sensing between electroporation electrodes 20 is additionally performed, in order to position the electrodes at the boundary of the tumor.

For some applications, and as illustrated in FIG. 9B, a device 109′ otherwise identical to device 109 has bioimpedance sensing electrodes 26 disposed on each distal region 19, in order to provide information regarding the position of each respective electroporation electrode 20 before applying the electroporation pulse. For example, each bioimpedance-sensing electrode 26 may be positioned proximally to an electroporation electrode 20 disposed on that distal region 19, such that identifying that bioimpedance-sensing electrodes 26 are disposed just outside of tumor 15 provides verification that electrodes 20 are just within the boundary of the tumor—e.g. as described hereinabove, mutatis mutandis.

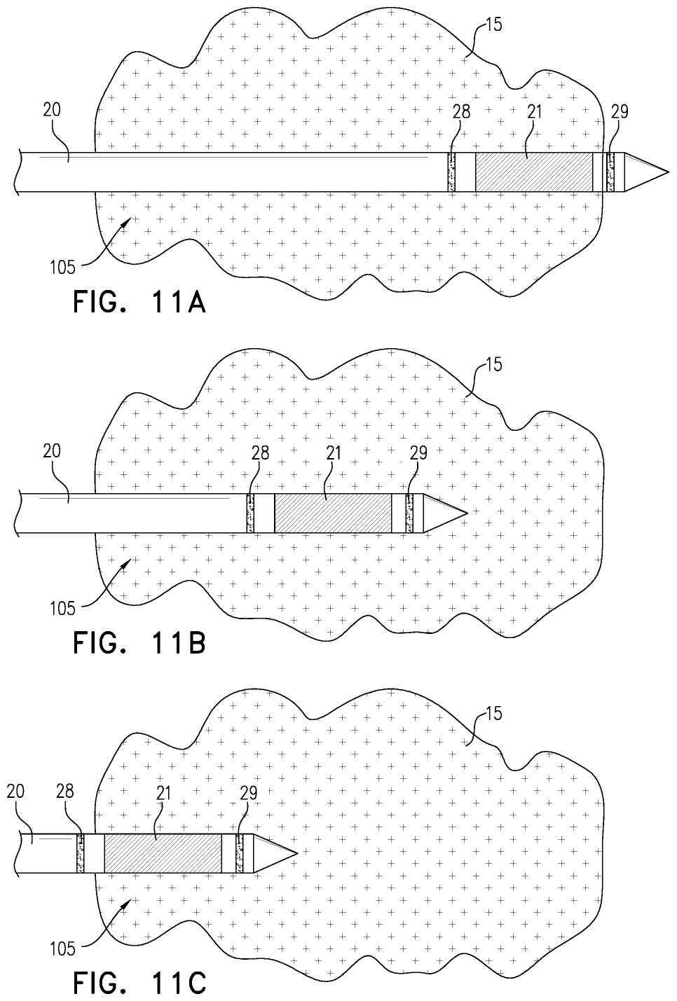

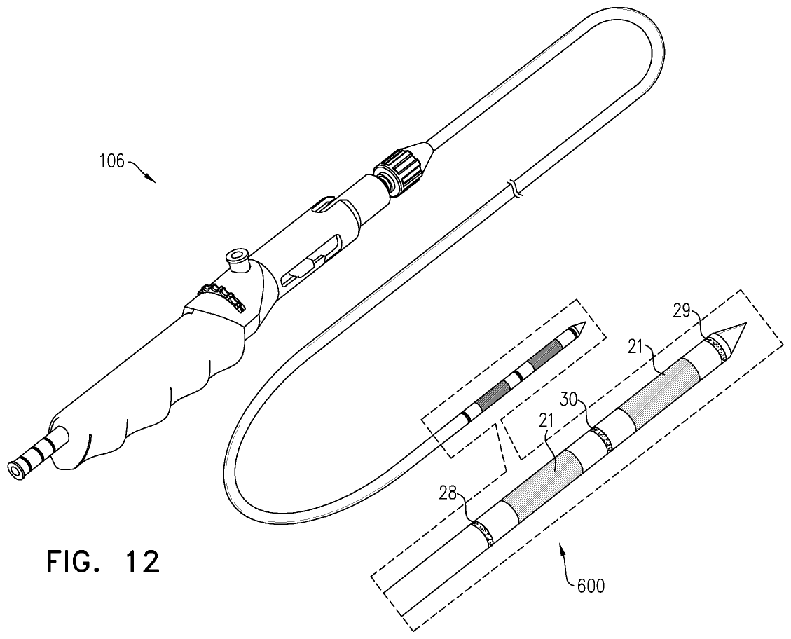

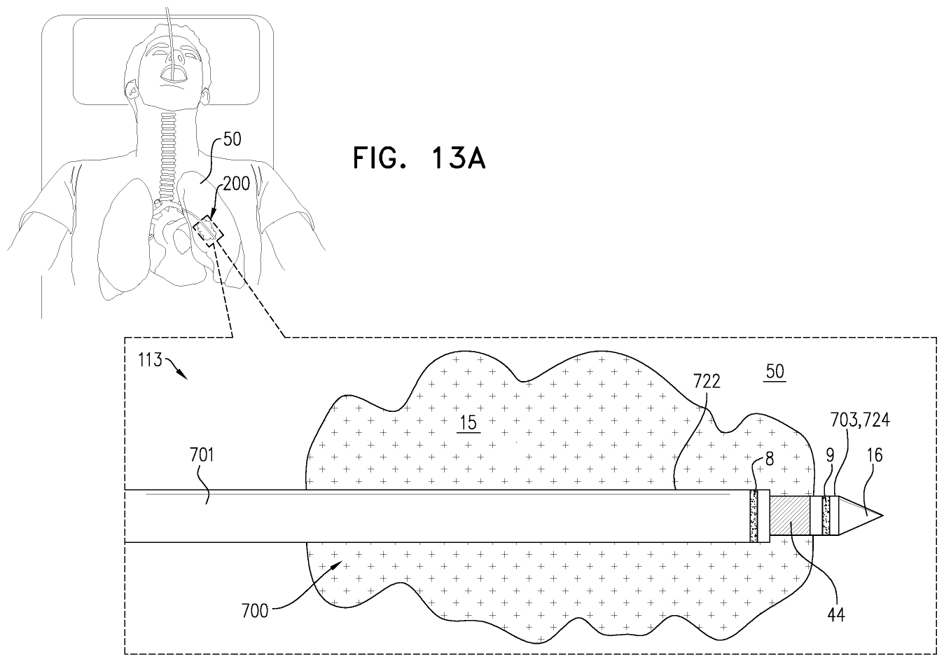

Reference is now made to FIGS. 10 and 11A-C which are schematic illustrations of a device 105, in accordance with some applications. Device 105 may be considered to be a variant of any of the devices mentioned hereinabove, and may be a part of a system such as system 2000, and/or can be compatible with a control unit such as control unit 2010. Similarly to the devices mentioned hereinabove, device 105 has a distal region 500, adapted to be advanced into a tumor. Unlike the devices mentioned hereinabove, device 105 may comprise a single electroporation electrode 21, which is typically flanked by a distal bioimpedance-sensing electrode 28 and a proximal bioimpedance-sensing electrode 29. A remote electrode (such as remote electrode 40 or similar, e.g. a skin electrode), may be used to facilitate delivery of an electroporation pulse to the tumor—e.g. with the remote electrode serving as a return electrode.

For some applications, the flanking of electroporation electrode 21 by bioimpedance-sensing electrodes 28 and 29 allows for the bioimpedance-sensing electrodes to be used by a control unit to detect the distal and/or proximal boundaries of the tumor, and to provide an alert indicating, for example, that no further distal or proximal advancement is required (or desired).

As illustrated by FIGS. 11A-C, distal region 500 can be advanced into a tumor 15 until bioimpedance-sensing electrode 29 exits the tumor—e.g. the distal boundary of the tumor (FIG. 11A). For example, a control unit may provide an alert to the operator in response to a change in bioimpedance detected via electrode 29 upon its exit from tumor 15. As described hereinabove with reference to FIGS. 6A-D, this positioning of bioimpedance-sensing electrode 29 just outside of the boundary of the tumor positions electroporation electrode 21 just within the boundary. Whilst electroporation electrode 21 is positioned at the boundary, an electroporation pulse may be applied to the tumor to ablate at least the surrounding portion of tumor.

FIG. 11B shows distal region 500 having been moved partway through the tumor (e.g. by withdrawing the distal region proximally). During the distal region's movement through the tumor, further electroporation pulses may be applied, in order to fully eradicate the tumor.

Bioimpedance-sensing electrode 28 can be used to detect the boundary of the tumor, and to alert the operator once the electroporation electrode has reached the opposite boundary (e.g. a proximal boundary) of the tumor (FIG. 11C). This may signify that electroporation of the tumor is complete.

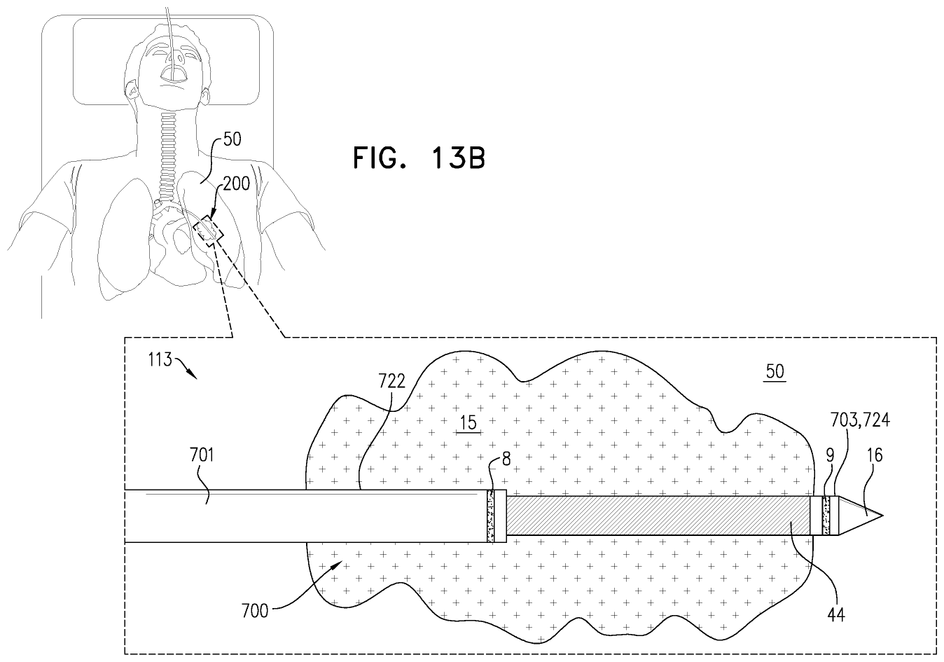

Reference is now made to FIG. 12, which illustrates a device 106, in accordance with some applications. Device 106 may be considered to be a variant of device 105, and may be a part of a system such as system 2000, and/or can be compatible with a control unit such as control unit 2010. Similarly to device 105, device 106 has a distal region 600, adapted to be advanced into a tumor. However, as shown in FIG. 12, device 106 comprises multiple (e.g. two) electroporation electrodes 21, such that, in some embodiments, an electroporation pulse may be applied in a bipolar mode between the electroporation electrodes.

For some applications, bioimpedance-sensing electrodes 28 and 29 are positioned at the distal and proximal ends of distal region 600, in order to identify boundaries of the tumor, as described hereinabove. For some applications, an additional bioimpedance-sensing electrode 30 is positioned between electroporation electrodes 21. Bioimpedance may be sensed between any combination of electrodes 28, 29, 30, and a remote electrode such as electrode 40.

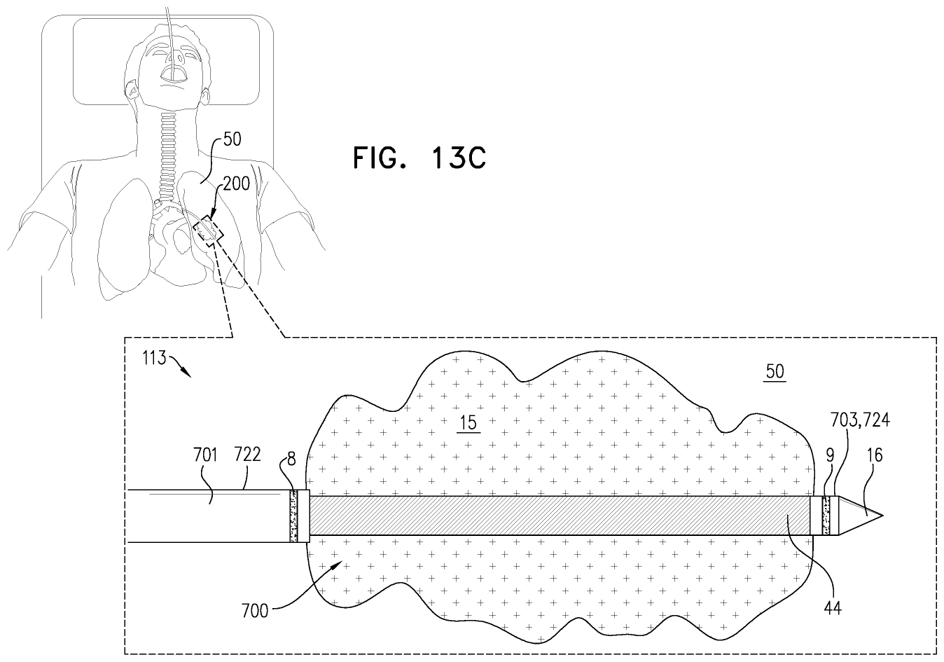

Reference is now made to FIGS. 13A-C, which are a schematic illustration of a device 113, in accordance with some applications.

Device 113 may be considered to be a variant of any of the devices mentioned hereinabove, and may be a part of a system such as system 2000, and/or can be compatible with a control unit such as control unit 2010. Similarly to the devices mentioned hereinabove, device 113 has a distal region 700, adapted to be advanced into a tumor. Device 113 comprises an electroporation electrode 44 at the distal region—e.g. may comprise only a single electroporation electrode. Device 113 is configured to facilitate adjustment of an effective length of electroporation electrode 44—e.g. according to a dimension of the tumor. A remote electrode (such as remote electrode 40 or similar, e.g. a skin electrode or a grounding pad), may be used to facilitate delivery of an electroporation pulse to the tumor—e.g. with the remote electrode serving as a return electrode.

For some such applications, distal region 700 includes a telescopic assembly—e.g. having a distal portion 724 and a proximal portion 722 which are axially slidable with respect to each other. For some such applications, device 113 comprises an outer shaft 701, and an inner shaft 703 that is adapted to slide into and/or out of a distal end of shaft 701. Shafts 701 and 703 may be coaxial. For some such applications, and as described with reference to devices 101 and 102 hereinabove, a distal region of shaft 703 serves as distal portion 724, and a distal region of shaft 701 serves as proximal portion 722.

For some applications, shaft 701 controls the effective length of electroporation electrode 44 (i.e. the length of the electroporation electrode that is exposed within the tissue), by insulating a portion of the electroporation electrode from the tissue. For such applications, shaft 701 (or at least the part of the shaft that serves as proximal portion 722) is typically electrically insulating—e.g. is formed from, or is coated in, a material that is an electrical insulator. For example, for some applications, electroporation electrode 44 is disposed on shaft 703, such that withdrawing shaft 701 proximally with respect to shaft 703 increases the effective length of the electroporation electrode by exposing more of the electroporation electrode, and advancing shaft 701 distally over shaft 703 decreases the effective length of the electroporation electrode by covering, and thus insulating, more of the electroporation electrode.

For some applications, a first bioimpedance-sensing electrode 8 is disposed on proximal portion 722, e.g. at a distal end of shaft 701, and a second bioimpedance-sensing electrode 9 is disposed on distal portion 724, e.g. distally to electrode 44. For such applications, such flanking of electroporation electrode 44 by the bioimpedance-sensing electrodes allows for the bioimpedance-sensing electrodes to be used by a control unit to detect the distal and/or proximal boundaries of the tumor and, for example, to thereby identify an appropriate length of distal region 700, and thereby an appropriate effective length of electrode 44—e.g. by providing an alert upon the appropriate length having been achieved. For some applications, and as shown, device 113 is configured such that, at the appropriate length, electroporation electrode 44 spans most of the length of the tumor (i.e. most of the distance between the distal boundary of the tumor and the proximal boundary of the tumor)—e.g. substantially the entire length of the tumor.

As illustrated by FIGS. 13A-C, distal region 700 can be advanced into a tumor 15 until bioimpedance-sensing electrode 9 exits the tumor—e.g. the distal boundary of the tumor (FIG. 13A). For example, a control unit may provide an alert to the operator in response to a change in bioimpedance detected via electrode 9 upon its exit from tumor 15. Similarly to as described hereinabove with reference to FIGS. 6A-D, this positioning of bioimpedance-sensing electrode 9 just outside of the boundary of the tumor positions the distal end of electroporation electrode 44 just within the boundary. In this state, electroporation electrode 44 may not yet have a desired effective length. The proximal portion 722 is thus then withdrawn proximally from distal portion 724 (e.g. by sliding shaft 701 proximally along shaft 703) thereby exposing more of electroporation electrode 44 within the tumor (FIG. 13B). In the example shown, proximal portion 722 is withdrawn until bioimpedance-sensing electrode 8 exits the tumor, and electroporation electrode 44 has the desired effective length—e.g. spanning substantially the entire length of the tumor (FIG. 13C). In this state, electroporation electrode 44 may then be driven (e.g. by power generator 7a or similar) to apply an electroporation pulse to the tumor.

For some applications, device 113 is adjustable in a manner that allows the effective length of electroporation electrode 44 to be at least 1 mm and/or no more than 50 mm.

Reference is now made to FIG. 4, which is a schematic cross-section through shaft 1, in accordance with some applications.

In some embodiments, hollow shaft 1 may be constructed in the form of a braided shaft where the outer jacket, 212, and inner liner, 214, are biocompatible polymers (such as Pebax, Silicone, Polyurethane, Polyethylene, and/or Teflon). Braid 13 is preferably metallic (for example Tungsten or stainless steel wires may be used). One or more of the braid wires may serve as the conductor that electrically connects electrode 2 and/or electrode 8 to their respective terminals. The internal diameter of hollow shaft 1 is typically at least 0.25 mm, such as at least 0.35 mm. The outer diameter is typically less than 5 mm, such as less than 2 mm. Using a flexible structure such as a braid is advantageous as it allows delivery of the device though complex anatomies. An example of this may be endo-bronchoscopic insertion of the device for treatment of lung tumor.

In some embodiments, hollow shaft 1 is constructed of additional layers of braid and polymer tubing. Advantageously, the additional layers may be used for delivering the signal from bioimpedance-sensing electrode 8. For example, braid 13 may serve as the conductor for electrode 2, whereas the braid of an additional layer may serve as the conductor for electrode 8.

In some embodiments the braid may be replaced with multiple wires electrically insulated from one another.

In some embodiments, any of the braids may be replaced with a hypotube. The hypotube may be laser cut to improve flexibility. In other embodiments, any of the braids may be replaced with a coil or a wire.

In some embodiments, shaft 3 may be constructed in a similar manner to hollow shaft 1, mutatis mutandis.

In some embodiments shaft 3 may be configured to include an inner lumen that may have a distal opening. In other embodiments the distal end may be closed—e.g. as shown. The outer diameter of shaft 3 is typically less than 2 mm, such as less than 1 mm.

In some embodiments, shaft 3 may be constructed from an insulated tube and the electric signal delivered from the proximal terminal to the electrode using a conductive wire passed through it. In some embodiments, it may be constructed from a metallic tube or multiple metallic tubes partly covered with a layer of insulation or multiple layers of insulation.

In some embodiments, the signal from the bioimpedance-sensing electrodes is delivered in a similar manner as described for driving the pules(s) to the electroporation electrodes. For example, an additional layer of braid, coil or tubing, insulated from the electroporation electrodes and their wiring, may be included.

Reference is again made to FIGS. 1-13C. For some applications, the same electrodes that are used for bioimpedance sensing are also used for electroporation—e.g. for such applications electrodes 2 and 4 illustrated in FIG. 1 are also used to provide control unit 1010 with information regarding the bioimpedance sensed at the tumor, in order to provide the control unit with information regarding the positioning of the electrodes with respect to the tumor.

It is to be understood that although reference has been made to electroporation electrodes throughout the application, for some applications another type of treatment applicator may be used. A nonlimiting list of possible treatment applicators includes RF electrodes, a radiation (e.g. microwave, laser) applicator, a high-intensity focused ultrasound transducer, or a dispenser for a drug (e.g. chemotherapy agent), a caustic agent (e.g. ethanol), a cryogenic fluid, or a radiation source. For some applications, such another treatment applicator is used instead of electroporation electrodes. For some applications, such another treatment is used in combination with electroporation electrodes—e.g. in order to provide a synergistic effect. For some such applications, a treatment applicator for such an additional treatment is coupled to and/or advanced through the outer shaft of the device. For other such applications, the treatment applicator for such an additional treatment is coupled to and/or advanced through the inner shaft of the device.

In some embodiments, the electroporation and/or bioimpedance-sensing electrodes may be constructed from biocompatible metals (such as Platinum, Iridium, Gold, Tungsten, Stainless Steel, Titanium).

In some embodiments the electroporation and/or bioimpedance-sensing electrodes may be in the form of a coil, braid, mesh, ring or laser cut tube.

In some embodiments, any of the bioimpedance-sensing elements may be in the form of a ring, wire, printed circuit.

In some embodiments, the length of each electroporation electrode may be at least 1 mm (e.g. at least 2 mm) and/or no more than 50 mm (e.g. no more than 20 mm, such as no more than 10 mm).

In some embodiments, the length of each bioimpedance-sensing electrode may be at least 1 mm (e.g. at least 2 mm) and/or no more than 50 mm (e.g. no more than 20 mm, e.g. no more than 10 mm, such as no more than 5 mm).

In some embodiments the different electroporation electrodes have different shapes or sizes to achieve optimal field distribution. For example one may be longer than the other.

In some embodiments, the diameter of electrode 4 may be at least 0.25 mm (e.g. at least 0.35 mm) and/or no more than 2 mm (e.g. no more than 1 mm).

In some embodiments, the diameter of electrode 2 may be at least 0.35 mm (e.g. at least 0.5 mm) and/or no more than 4 mm (e.g. no more than 2 mm).

Distance L, defined as the distance between the proximal end of inner electrode 4 and distal end of outer electrode 2, may be adjustable to enable coverage of the target tissue and optimal energy distribution. In some embodiments, the apparatus is configured such that the maximal value of L is at least 5 mm, for example at least 20 mm.

In some embodiments, power generator 7 is configured to apply high voltage pulses required for electroporation. For example, each pulse may have a duration of at least 0.1 microseconds (e.g. at least 0.5 microseconds, e.g. at least 1 microsecond, such as at least 2 microseconds) and/or no more than 1 second—e.g. no more than 1 millisecond, e.g. no more than 0.5 milliseconds, e.g. no more than 100 microseconds, such as no more than 10 microseconds, such as no more than 2 microseconds (e.g. 0.1-100 microseconds). The pulses may be temporally spaced apart by at least 0.1 microseconds (e.g. at least 1 microsecond) and/or no more than 1 millisecond, e.g. no more than 0.5 milliseconds, e.g. no more than 100 microseconds, such as no more than 10 microseconds, such as no more than 2 microseconds (e.g. 0.1-10 microseconds).

The electrical current of each pulse may have a frequency of 1-5 MHZ, e.g. 1-3 MHz, such as 1-2.5 MHz. The electrical current used may have a voltage sufficient to induce either reversible or irreversible electroporation. For some applications, each pulse has a voltage of at least 200 V (e.g. at least 500 V, such as at least 2000 V) and/or no more than 1000 V (e.g. no more than 500 V)—e.g. 200-500 V. In some embodiments, the polarity of the voltage may alternate between positive and negative polarities (e.g. bipolar mode), or alternatively the voltage may not alternate polarity (e.g. monopolar mode).

For some applications, the pulses may be applied in multiple pulse trains (within which the pulses may have characteristics and spacing described hereinabove) with a rest period therebetween. For some applications, each train may consist of at least 100 pulses (e.g. at least 1,000 pulses) and/or no more than 1 million pulses (e.g. no more than 100,000 pulses, e.g. no more than 10,000 pulses, such as no more than 1,000 pulses). For some applications, a rest period of at least 100 milliseconds (e.g. at least 500 milliseconds, such as at least 1 second) and/or less than 1 minute (e.g. less than 10 seconds, such as less than 5 seconds) is provided between pulse trains.

Alternatively, the power generator may be used to drive one or more of the electrode of the device to apply RF energy for RF ablation—e.g. using alternating currents of 300-600 kHz.

In some embodiments, microwave antennas may be used instead of the electroporation electrodes and power generator 7 may be a microwave generator.

In some embodiments, the electrodes are configured to be delivered percutaneously (e.g. transluminally). The effective length of such a percutaneous device, described as the length between the proximal terminal and electrode may be for example at least 10 cm and/or no more than 50 cm, such as between 15 cm and 50 cm.

In some embodiments, the electrodes are configured to be delivered through a bronchoscope. The effective length of the device, described as the length between the proximal terminal and electrode, may be for example at least 40 cm, such as between 60 cm and 150 cm. Endobronchial access has the advantage of reducing potential safety risks (such as pneumothorax).

In some embodiments, the electrodes are configured to be delivered through a sheath or catheter. In such embodiments the effective length of the device, described as the length between the proximal terminal and electrode may be for example at least 40 cm, such as between 60 cm and 180 cm.

In some embodiments the bioimpedance-sensing electrodes may be further used to evaluate the electric field generated during the ablation process. This may be of specific use when using electroporation for ablation. The electric field measured and the impedance values may be used to assess the ablation area or adjust the applied voltage accordingly.

In some embodiments, any of the shafts described herein may be used to biopsy the target site (e.g. the tumor)—e.g. guided by bioimpedance-sensing. For example, one or more of the shafts described herein may be coupled to, may be configured to guide, and/or may comprise a biopsy tool such as a biopsy needle.

For some applications, bioimpedance sensing is performed as part of a diagnostic procedure. For some applications, the bioimpedance-sensing electrodes may be used to determine whether a lesion or growth is a cancerous tumor—e.g. to facilitate diagnosis. For example, the bioimpedance of a cancerous tumor may be different to that of a non-cancerous lesion or growth.

For some applications, the device may be used to perform a biopsy in addition to using bioimpedance sensing to determine whether a tumor is cancerous.

For some applications, it may be possible to determine whether the bioimpedance-sensing electrode(s) (and therefore the electroporation electrode(s)) are positioned within the tumor by monitoring the bioimpedance measured at the bioimpedance-sensing electrode(s) over the breathing cycle of the patient. For example, the bioimpedance values of the parenchyma of the lung may vary (e.g. oscillate) during the breathing cycle (e.g. impedance decreasing during exhalation and increasing during inhalation) whereas a tumor may have a more consistent bioimpedance value throughout the breathing cycle. For some applications, receiving a signal with a magnitude of oscillation above a threshold magnitude is indicative that the tissue in which the bioimpedance-sensing electrode is disposed is lung tissue (e.g. parenchyma of the lung) rather than tumor tissue.

For some applications, the frequency of the oscillations sensed at the tumor by the bioimpedance-sensing electrode is compared with the frequency of the breathing cycle of the patient to ensure that the sensed oscillations are reflective of the breathing cycle, and do not arise from other factors (e.g. the pulse of the patient). This may therefore provide further validation to the operator regarding whether a particular tissue is a tumor, and/or the position of the bioimpedance-sensing electrode(s) with respect to the tumor.

There is therefore provided, in accordance with some applications, a method comprising:
receiving information indicative of bioimpedance of a tissue in a lung of a subject (optionally including information indicative of oscillation of the bioimpedance, such as magnitude and/or frequency of the oscillation); and
responsively, determining (e.g. diagnosing) whether the tissue is a tumor and/or is cancerous.

Tumor ablation is given as an example for one use of the device, however any of the described above may be used also for inducing electric fields for the enhancement of drug uptake or for ablating other target tissues or sites.

It is to be noted that, for some applications, any of the skin electrodes described herein may be replaced with a grounding pad.

It is to be noted that, although the electrodes (e.g. the bioimpedance-sensing electrodes and the electroporation electrodes) described herein are shown as rings or tubes that circumscribe their respective shafts, the scope of the present disclosure includes other shapes and arrangements.

It will be appreciated by persons skilled in the art that the present disclosure is not limited to what has been particularly shown and described hereinabove. Rather, the scope of embodiments of the present disclosure includes both combinations and sub combinations of the various features described hereinabove, as well as variations and modifications thereof that are not in the prior art, which would occur to persons skilled in the art upon reading the foregoing description.

1-123. (canceled)

124. Apparatus for use with a tumor within a subject, the apparatus comprising a tumor-ablating device that has a distal region that comprises:
an inner shaft, comprising:
a first bioimpedance-sensing electrode;
a first electroporation electrode, mounted at a fixed position proximally from the first bioimpedance-sensing electrode; and
an outer shaft within which the inner shaft is coaxially disposed, the outer shaft comprising:
a second bioimpedance-sensing electrode; and
a second electroporation electrode, mounted at a fixed position distally from the second bioimpedance-sensing electrode,
wherein the inner shaft and the outer shaft are telescopically slidable with respect to each other in a manner that changes a first axial distance between the first electroporation electrode and the second electroporation electrode.

125. The apparatus according to claim 124, wherein the apparatus is for use with a tumor in a lung of the subject, and wherein the distal region is transbronchially deliverable to the lung.

126. The apparatus according to claim 124, wherein the tumor has a boundary, and wherein, via the telescopic sliding of the inner shaft and the outer shaft with respect to each other, the distal region is advanceable into a position within the lung in which:
both the first and the second electroporation electrodes are disposed within the boundary of the tumor, and
both the first and second bioimpedance-sensing electrodes are disposed outside of the boundary of the tumor, on opposite sides of the tumor to one another.

127. The apparatus according to claim 124, wherein the apparatus is configured to sense bioimpedance at the tumor by sensing:
bioimpedance between the first electroporation electrode and the first bioimpedance-sensing electrode, and
bioimpedance between the second electroporation electrode and the second bioimpedance-sensing electrode.

128. The apparatus according to claim 124, wherein the apparatus is configured to sense bioimpedance at the tumor by sensing bioimpedance between the first bioimpedance-sensing electrode and the second bioimpedance-sensing electrode.

129. The apparatus according to claim 124, wherein the inner shaft is advanceable out of a distal end of the outer shaft.

130. The apparatus according to claim 124, wherein the outer shaft defines a side-port out of which the inner shaft is advanceable.

131. The apparatus according to claim 124, wherein the inner shaft is curvable with respect to the outer shaft.

132. The apparatus according to claim 124, the apparatus further comprises a control unit that comprises a power generator, electrically connectable to both the first and second electroporation electrodes, and adapted to drive an electroporation pulse therebetween.

133. The apparatus according to claim 124, wherein the outer shaft is formed from an electrical insulator.

134. The apparatus according to claim 133, wherein the device is configured such that an effective length of the first electroporation electrode is adjustable by sliding the outer shaft over the first electroporation electrode.

135. The apparatus according to claim 124, wherein the apparatus comprises a control unit, adapted to receive signals from:
the first bioimpedance-sensing electrode, and to responsively provide an output indicative of bioimpedance of tissue adjacent the first electroporation electrode, and
the second bioimpedance-sensing electrode, and to responsively provide an output indicative of bioimpedance of tissue adjacent the second electroporation electrode.

136. The apparatus according to claim 135, wherein the control unit is configured to, responsively to the signals, output information indicative of a position of each of the first electroporation electrode and the second electroporation electrode with respect to the tumor.

137. The apparatus according to claim 135, wherein the control unit is configured to, responsively to the signals:
determine whether the distal region is in a state in which the first electroporation electrode and the second electroporation electrode are both disposed inside the tumor, and
responsively to determining that the distal region is in the state, provide an indication that the first electroporation electrode and the second electroporation electrode are both disposed inside the tumor.

138. The apparatus according to claim 135, wherein the control unit is adapted to generate the signals by driving an electrical pulse between the first and second bioimpedance-sensing electrodes.

139. The apparatus according to claim 135, wherein the control unit is adapted to receive the signals by:
driving a first electrical pulse between the first electroporation electrode and the first bioimpedance-sensing electrode, and
driving a second electrical pulse between the second electroporation electrode and the second bioimpedance-sensing electrode.

140. The apparatus according to claim 135, wherein the control unit is configured to, responsively to the signals, output information indicative of a position of the first bioimpedance-sensing electrode and the second bioimpedance-sensing electrode with respect to the tumor.

141. The apparatus according to claim 140, wherein the control unit is adapted to:
responsively to the signals, differentiate a state of the distal region in which the first and second bioimpedance-sensing electrodes are both positioned outside of the tumor, from one or more other states of the distal region in which at least one of the first and second bioimpedance-sensing electrodes is positioned inside of the tumor, and
responsively to the differentiation, provide an indication that the distal region is in the state.

142. The apparatus according to claim 140, wherein the control unit is adapted to:
responsively to the signals, differentiate between (i) a state of the distal region in which the first and second bioimpedance-sensing electrodes are both positioned outside of the tumor, and (ii) one or more other states of the distal region in which at least one of the first and second bioimpedance-sensing electrodes is positioned inside of the tumor, and
responsively to the differentiation, provide an indication of whether the first and second electroporation electrodes are positioned within the tumor.

143. The apparatus according to claim 124, wherein:
the tumor is a tumor disposed in a lung of a subject, and
the apparatus is configured to determine a boundary of the lung tumor via sensing of bioimpedance via at least one of the first and the second bioimpedance-sensing electrodes.

144. The apparatus according to claim 143, wherein the apparatus comprises a bronchoscope, transbronchially advanceable to the lung, the distal region being deliverable to the tumor via the bronchoscope.

145. A method for ablating a tumor in a tissue of a subject, the method comprising:
advancing a distal region of a tumor-ablating device into the tissue, the distal region including:
a first shaft, including:
a first bioimpedance-sensing electrode; and
a first electroporation electrode, mounted at a fixed position with respect to the first bioimpedance-sensing electrode; and
a second shaft, including:
a second bioimpedance-sensing electrode; and
a second electroporation electrode, mounted at a fixed position with respect to the second bioimpedance-sensing electrode,
by sensing bioimpedance of the tissue using at least one of the first and second bioimpedance-sensing electrodes, identifying a boundary of the tumor;
responsively to the identifying, positioning the first electroporation electrode and the second electroporation electrode within the boundary by telescopically sliding the first shaft with respect to the second shaft; and
while the first and second electroporation electrodes remain within the boundary, applying an electroporation pulse to the tumor by driving the electroporation pulse between the first and second electroporation electrodes.
